(19) United States
(12) Patent Application Publication
(10) Pub. No.: US 2026/0284279 A1
(43) Pub. Date: Sep. 24, 2026

(54) MEDICAL DEVICE COATINGS AND METHODS

(71) Applicant: Surmodics, Inc., Eden Prairie, MN (US)

(72) Inventors: David E. Babcock, Brooklyn Park, MN (US); Joram Slager, St. Louis Park, MN (US); Syed Hossainy, Hayward, CA (US); Ralph A. Chappa, Ham Lake, MN (US)

(21) Appl. No.: 19/368,473

(22) Filed: Oct. 24, 2025

Related U.S. Application Data
(60) Provisional application No. 63/712,040, filed on Oct. 25, 2024.

(51) Int. Cl. A61L 31/10; A61L 33/08

(52) U.S. Cl. CPC A61L 31/10; A61L 33/08; A61L 2420/02; A61L 2420/08

(57) ABSTRACT

Embodiments herein include multilayer thromboresistant coatings and medical devices including the same. In various embodiments, the thromboresistant coating can include a medical device with an antithrombogenic coating comprising a substrate, a tie layer disposed over the substrate, a hydrophilic layer disposed over the tie layer, and a heparin layer disposed over the hydrophilic layer. The hydrophilic layer comprises a photo-PVP compound and the heparin layer comprises a photo-heparin. The tie layer, the hydrophilic layer, and the heparin layer are less than or equal to 100 nm thick in total. Other embodiments are also included herein.

This application claims the benefit of U.S. Provisional Application No. 63/712,040, filed Oct. 25, 2024, the content of which is herein incorporated by reference in its entirety.

### FIELD

Embodiments herein relate to medical devices and coatings for the same.

### BACKGROUND

Medical devices include those that are chronically implanted, devices that are transitorily implanted, and those that are not implanted at all but in contact with tissue and/or bodily fluids, amongst others. Many types of medical devices can be enhanced by coatings that provide various useful properties to the surfaces of medical device.

Intravascular devices, such as catheters and guidewires, are inserted into the vascular system and navigated through the vasculature to reach treatment sites within the vascular system. Certain devices may only be briefly exposed to blood (i.e., catheters, guidewires) while other devices (i.e., blood filters, stents) may remain in the blood longer. However, even brief exposure to blood can trigger proteins in the blood to begin attaching or “fouling” the surfaces of the device or implant. These attached proteins can trigger adhesion of platelets or other matter to the device or implant surfaces, which can cause thrombosis or clotting of the device or implant. Over time, these clots can grow and eventually occlude the vessel. Thrombogenicity refers to the tendency of a material in contact with the blood to produce a thrombus, or clot. Further, the attached proteins can attract bacteria that can form biofilms on the device or implant surfaces, which can lead to infections, inflammation, or other health issues.

Coatings with thromboresistant properties can be useful with some types of medical devices. For example, thromboresistant coatings can reduce or eliminate thrombogenicity and issues with biofouling.

### SUMMARY

Embodiments herein relate to medical devices and coatings for the same. In a first aspect, a medical device with an antithrombogenic coating can be included having a substrate, a tie layer, wherein the tie layer can be disposed over the substrate, and a hydrophilic layer, wherein the hydrophilic layer can be disposed over the tie layer, the hydrophilic layer can include a photo-PVP, a heparin layer, wherein the heparin layer can be disposed over the hydrophilic layer, the heparin layer can include a photo-heparin, and wherein the tie layer, the hydrophilic layer, and the heparin layer can be less than or equal to 100 nm thick in total.

In a second aspect, in addition to one or more of the preceding or following aspects, or in the alternative to some aspects, the substrate can be a metal or a polymer.

In a third aspect, in addition to one or more of the preceding or following aspects, or in the alternative to some aspects, the tie layer can include at least one selected from the group consisting of a silane compound and a phosphonate compound.

In a fourth aspect, in addition to one or more of the preceding or following aspects, or in the alternative to some aspects, the photo-PVP can include acetylated PVP-APMA-BBA.

In a fifth aspect, in addition to one or more of the preceding or following aspects, or in the alternative to some aspects, the hydrophilic layer can further include sodium bis(4-benzoylphenyl phosphate) and acetylated PVP-APMA-BBA.

In a sixth aspect, in addition to one or more of the preceding or following aspects, or in the alternative to some aspects, the heparin layer includes at least two coats or at least three coats of the photo-heparin.

In a seventh aspect, in addition to one or more of the preceding or following aspects, or in the alternative to some aspects, the tie layer can include 1,4-bis(trimethoxysilylethyl)benzene.

In an eighth aspect, in addition to one or more of the preceding or following aspects, or in the alternative to some aspects, the medical device can be a neurovascular stent.

In a ninth aspect, a medical device with an antithrombogenic coating can be included having a substrate, and a hydrophilic layer, the hydrophilic layer can include a photo-PVP, a heparin layer, wherein the heparin layer can be disposed over the hydrophilic layer, the heparin layer can include a photo-heparin, and wherein the hydrophilic layer and the heparin layer can be less than or equal to 100 nm thick in total.

In a tenth aspect, in addition to one or more of the preceding or following aspects, or in the alternative to some aspects, the substrate can be a metal or a polymer.

In an eleventh aspect, in addition to one or more of the preceding or following aspects, or in the alternative to some aspects, the photo-PVP can include acetylated PVP-APMA-BBA.

In a twelfth aspect, in addition to one or more of the preceding or following aspects, or in the alternative to some aspects, the hydrophilic layer can further include sodium bis(4-benzoylphenyl phosphate) and acetylated PVP-APMA-BBA.

In a thirteenth aspect, in addition to one or more of the preceding or following aspects, or in the alternative to some aspects, the heparin layer includes at least two coats or at least three coats of the photo-heparin.

In a fourteenth aspect, in addition to one or more of the preceding or following aspects, or in the alternative to some aspects, the medical device can be a neurovascular stent.

In a fifteenth aspect, a medical device with an antithrombogenic coating can be included having a substrate, a tie layer, wherein the tie layer can be disposed over the substrate, and a first hydrophilic layer, wherein the first hydrophilic layer can be disposed over the tie layer, the first hydrophilic layer can include a first photo-PVP, a first heparin layer, wherein the first heparin layer can be disposed over the first hydrophilic layer, the first heparin layer can include a first photo-heparin, a second hydrophilic layer, the second hydrophilic layer can include a second photo-PVP, wherein the second hydrophilic layer can be disposed over the first heparin layer, a second heparin layer, the second heparin layer can include a second photo-heparin, wherein the second heparin layer can be disposed over the second hydrophilic layer, and wherein the tie layer, the first hydrophilic layer, the first heparin layer, the second hydrophilic layer, and the second heparin layer can be less than or equal to 100 nm thick in total.

In a sixteenth aspect, in addition to one or more of the preceding or following aspects, or in the alternative to some aspects, the substrate can be a metal or a polymer.

In a seventeenth aspect, in addition to one or more of the preceding or following aspects, or in the alternative to some aspects, the tie layer can include at least one selected from the group consisting of a silane compound and a phosphonate compound.

In an eighteenth aspect, in addition to one or more of the preceding or following aspects, or in the alternative to some aspects, the first photo-PVP and the second photo-PVP include acetylated PVP-APMA-BBA.

In a nineteenth aspect, in addition to one or more of the preceding or following aspects, or in the alternative to some aspects, the tie layer can include 1,4-bis(trimethoxysilylethyl)benzene.

In a twentieth aspect, in addition to one or more of the preceding or following aspects, or in the alternative to some aspects, the medical device can be a neurovascular stent.

In a twenty-first aspect, a medical device with an antithrombogenic coating can be included having a substrate, a tie layer, wherein the tie layer can be disposed over the substrate, and a hydrophilic layer, wherein the hydrophilic layer can be disposed over the tie layer, the hydrophilic layer can include a photo-PVP, a heparin and PVP layer, wherein the heparin and PVP layer can be disposed over the hydrophilic layer, the heparin and PVP layer can include a photo-heparin, and a photo-PVP, and wherein the tie layer, the hydrophilic layer, and the heparin and PVP layer can be less than or equal to 100 nm thick in total.

In a twenty-second aspect, in addition to one or more of the preceding or following aspects, or in the alternative to some aspects, the substrate can be a metal or a polymer.

In a twenty-third aspect, in addition to one or more of the preceding or following aspects, or in the alternative to some aspects, the tie layer can include at least one selected from the group consisting of a silane compound and a phosphonate compound.

In a twenty-fourth aspect, in addition to one or more of the preceding or following aspects, or in the alternative to some aspects, the photo-PVP can include acetylated PVP-APMA-BBA.

In a twenty-fifth aspect, in addition to one or more of the preceding or following aspects, or in the alternative to some aspects, the tie layer can include 1,4-bis(trimethoxysilylethyl)benzene.

In a twenty-sixth aspect, in addition to one or more of the preceding or following aspects, or in the alternative to some aspects, the medical device can be a neurovascular stent.

In a twenty-seventh aspect, a medical device with an antithrombogenic coating can be included having a substrate, a tie layer, wherein the tie layer can be disposed over the substrate, and a hydrophilic layer, wherein the hydrophilic layer can be disposed over the tie layer, the hydrophilic layer can include a photo-PVP, and iodine, a heparin layer, wherein the heparin layer can be disposed over the hydrophilic layer, the heparin layer can include a photo-heparin, and wherein the tie layer, the hydrophilic layer, and the heparin layer can be less than or equal to 100 nm thick in total.

In a twenty-eighth aspect, in addition to one or more of the preceding or following aspects, or in the alternative to some aspects, the substrate can be a metal or a polymer.

In a twenty-ninth aspect, in addition to one or more of the preceding or following aspects, or in the alternative to some aspects, the tie layer can include at least one selected from the group consisting of a silane compound and a phosphonate compound.

In a thirtieth aspect, in addition to one or more of the preceding or following aspects, or in the alternative to some aspects, the photo-PVP can include acetylated PVP-APMA-BBA.

In a thirty-first aspect, in addition to one or more of the preceding or following aspects, or in the alternative to some aspects, the tie layer can include 1,4-bis(trimethoxysilylethyl)benzene.

In a thirty-second aspect, in addition to one or more of the preceding or following aspects, or in the alternative to some aspects, the heparin layer includes at least two coats of the photo-heparin.

In a thirty-third aspect, in addition to one or more of the preceding or following aspects, or in the alternative to some aspects, the medical device can be a neurovascular stent.

In a thirty-fourth aspect, a medical device with an antithrombogenic coating can be included having a substrate, a tie layer, wherein the tie layer can be disposed over the substrate, and a hydrophilic layer, wherein the hydrophilic layer can be disposed over the tie layer, the hydrophilic layer can include a photo-PVP, a heparin layer, wherein the heparin layer can be disposed over the hydrophilic layer, the heparin layer can include a photo-heparin, and a second hydrophilic layer, wherein the second hydrophilic layer can be disposed over the heparin layer, the second hydrophilic layer can include a second photo-PVP.

In a thirty-fifth aspect, in addition to one or more of the preceding or following aspects, or in the alternative to some aspects, the second hydrophilic layer can be deposited by applying UV radiation while the medical device can be immersed in a solution of the second photo-PVP and a solvent.

In a thirty-sixth aspect, in addition to one or more of the preceding or following aspects, or in the alternative to some aspects, a solution of the second photo-PVP and a solvent can be between 0 mg/ml and 10 mg/ml.

In a thirty-seventh aspect, in addition to one or more of the preceding or following aspects, or in the alternative to some aspects, the photo-PVP can include acetylated PVP-APMA-BBA.

In a thirty-eighth aspect, in addition to one or more of the preceding or following aspects, or in the alternative to some aspects, the second photo-PVP can include PVP-APMA-BBA.

In a thirty-ninth aspect, in addition to one or more of the preceding or following aspects, or in the alternative to some aspects, the tie layer can include at least one selected from the group consisting of a silane compound and a phosphonate compound.

In a fortieth aspect, in addition to one or more of the preceding or following aspects, or in the alternative to some aspects, the tie layer can include 1,4-bis(trimethoxysilylethyl)benzene.

In a forty-first aspect, in addition to one or more of the preceding or following aspects, or in the alternative to some aspects, the heparin layer includes at least two coats or at least three coats of the photo-heparin.

In a forty-second aspect, in addition to one or more of the preceding or following aspects, or in the alternative to some aspects, the tie layer, the hydrophilic layer, the heparin layer, and the second hydrophilic layer can be less than or equal to 100 nm thick in total.

In a forty-third aspect, in addition to one or more of the preceding or following aspects, or in the alternative to some aspects, the substrate can be a metal or a polymer. In a forty-fourth aspect, in addition to one or more of the preceding or following aspects, or in the alternative to some aspects, the medical device can be a neurovascular stent.

In a forty-fifth aspect, in addition to one or more of the preceding or following aspects, or in the alternative to some aspects, the coating exhibits enhanced heparin activity and low fibrinogen binding.

In a forty-sixth aspect, a medical device with an antithrombogenic coating can be included having a substrate, and a hydrophilic layer, the hydrophilic layer can include a photo-PVP, a heparin layer, wherein the heparin layer can be disposed over the hydrophilic layer, the heparin layer can include a photo-heparin, and a second hydrophilic layer, wherein the second hydrophilic layer can be disposed over the heparin layer, the second hydrophilic layer can include a second photo-PVP.

In a forty-seventh aspect, in addition to one or more of the preceding or following aspects, or in the alternative to some aspects, the second hydrophilic layer can be deposited by applying UV radiation while the medical device can be immersed in a solution of the second photo-PVP and a solvent.

In a forty-eighth aspect, in addition to one or more of the preceding or following aspects, or in the alternative to some aspects, a solution of the second photo-PVP and a solvent can have a concentration of the second photo-PVP of between 0 mg/ml and 10 mg/ml.

In a forty-ninth aspect, in addition to one or more of the preceding or following aspects, or in the alternative to some aspects, the photo-PVP can include acetylated PVP-APMA-BBA.

In a fiftieth aspect, in addition to one or more of the preceding or following aspects, or in the alternative to some aspects, the photo-PVP can include PVP-APMA-BBA.

In a fifty-first aspect, in addition to one or more of the preceding or following aspects, or in the alternative to some aspects, the second photo-PVP can include PVP-APMA-BBA.

In a fifty-second aspect, in addition to one or more of the preceding or following aspects, or in the alternative to some aspects, the heparin layer includes at least two coats of the photo-heparin.

In a fifty-third aspect, in addition to one or more of the preceding or following aspects, or in the alternative to some aspects, the hydrophilic layer, the heparin layer, and the second hydrophilic layer can be less than or equal to 100 nm thick in total.

In a fifty-fourth aspect, in addition to one or more of the preceding or following aspects, or in the alternative to some aspects, the substrate can be a polymer.

In a fifty-fifth aspect, in addition to one or more of the preceding or following aspects, or in the alternative to some aspects, the medical device can be a neurovascular stent.

In a fifty-sixth aspect, a medical device with an antithrombogenic coating can be included having a substrate, a tie layer, wherein the tie layer can be disposed over the substrate, and a hydrophilic layer, wherein the hydrophilic layer can be disposed over the tie layer, the hydrophilic layer can include a photo-PVP, a functional layer with heparin activity, wherein the functional layer with heparin activity can be disposed over the hydrophilic layer, the functional layer with heparin activity can include a compound with heparin activity, and wherein the tie layer, the hydrophilic layer, and the functional layer with heparin activity can be less than or equal to 100 nm thick in total.

In a fifty-seventh aspect, in addition to one or more of the preceding or following aspects, or in the alternative to some aspects, the compound with heparin activity can include at least one of a heparin-mimicking polymer and a heparin-mimicking polypeptide.

In a fifty-eighth aspect, in addition to one or more of the preceding or following aspects, or in the alternative to some aspects, the substrate can be a metal or a polymer.

In a fifty-ninth aspect, in addition to one or more of the preceding or following aspects, or in the alternative to some aspects, the tie layer can include at least one selected from the group consisting of a silane compound and a phosphonate compound.

In a sixtieth aspect, in addition to one or more of the preceding or following aspects, or in the alternative to some aspects, the photo-PVP can include acetylated PVP-APMA-BBA.

In a sixty-first aspect, in addition to one or more of the preceding or following aspects, or in the alternative to some aspects, the hydrophilic layer can further include sodium bis(4-benzoylphenyl phosphate) and acetylated PVP-APMA-BBA.

In a sixty-second aspect, in addition to one or more of the preceding or following aspects, or in the alternative to some aspects, the functional layer with heparin activity includes at least two coats of the compound with heparin activity.

In a sixty-third aspect, in addition to one or more of the preceding or following aspects, or in the alternative to some aspects, the tie layer can include 1,4-bis(trimethoxysilylethyl)benzene.

In a sixty-fourth aspect, in addition to one or more of the preceding or following aspects, or in the alternative to some aspects, the medical device can be a neurovascular stent.

In a sixty-fifth aspect, a medical device with an antithrombogenic coating can be included having a substrate, and a hydrophilic layer, the hydrophilic layer can include a photo-PVP, a heparin layer, wherein the heparin layer can be disposed over the hydrophilic layer, a photoreactive crosslinking agent, wherein the photoreactive crosslinking agent can be disposed within the hydrophilic layer and/or between the hydrophilic layer and the heparin layer, and wherein the hydrophilic layer and the heparin layer can be less than or equal to 100 nm thick in total.

In a sixty-sixth aspect, in addition to one or more of the preceding or following aspects, or in the alternative to some aspects, the substrate can be a metal or a polymer.

In a sixty-seventh aspect, in addition to one or more of the preceding or following aspects, or in the alternative to some aspects, the photo-PVP can include acetylated PVP-APMA-BBA.

In a sixty-eighth aspect, in addition to one or more of the preceding or following aspects, or in the alternative to some aspects, the hydrophilic layer can further include sodium bis(4-benzoylphenyl phosphate) and acetylated PVP-APMA-BBA.

In a sixty-ninth aspect, in addition to one or more of the preceding or following aspects, or in the alternative to some aspects, the heparin layer includes at least two coats of a non-photoreactive heparin compound.

In a seventieth aspect, in addition to one or more of the preceding or following aspects, or in the alternative to some aspects, the heparin layer can include a non-photoreactive heparin compound.

In a seventy-first aspect, in addition to one or more of the preceding or following aspects, or in the alternative to some aspects, the medical device can be a neurovascular stent.

In a seventy-second aspect, a method of depositing a coating on a medical device can be included. The method can include depositing a hydrophilic layer over a substrate using an in-solution coating technique, wherein the hydrophilic layer includes a photo-PVP compound, and depositing a heparin layer over the hydrophilic layer, wherein the heparin layer includes a photo-heparin, and wherein the total thickness of coating layers can be 100 nanometers or less.

In a seventy-third aspect, in addition to one or more of the preceding or following aspects, or in the alternative to some aspects, the method can further include depositing a tie layer over the substrate prior to depositing the hydrophilic layer.

In a seventy-fourth aspect, in addition to one or more of the preceding or following aspects, or in the alternative to some aspects, the substrate includes a metal or a polymer.

In a seventy-fifth aspect, in addition to one or more of the preceding or following aspects, or in the alternative to some aspects, the in-solution coating technique includes immersing the substrate in a solution of the photo-PVP compound at a concentration of less than 20 mg/ml and applying UV radiation while the substrate can be immersed.

In a seventy-sixth aspect, in addition to one or more of the preceding or following aspects, or in the alternative to some aspects, the concentration of the photo-PVP compound in the solution can be from 0.5 to 15 mg/ml.

In a seventy-seventh aspect, in addition to one or more of the preceding or following aspects, or in the alternative to some aspects, wherein depositing the heparin layer over the hydrophilic layer can be performed using a dip coating technique with a solution of the photo-heparin at a concentration of 50 to 100 mg/ml.

In a seventy-eighth aspect, in addition to one or more of the preceding or following aspects, or in the alternative to some aspects, the heparin layer can be applied in multiple coats.

In a seventy-ninth aspect, a method of depositing a coating on a medical device can be included. The method can include depositing a first hydrophilic layer over a substrate using an in-solution coating technique, wherein the first hydrophilic layer includes a photo-PVP compound, depositing a heparin layer over a hydrophilic layer, wherein the heparin layer includes a photo-heparin, and depositing a second hydrophilic layer using an in-solution coating technique, wherein the second hydrophilic layer includes a photo-PVP compound, and wherein the total thickness of coating layers can be 100 nanometers or less.

In an eightieth aspect, in addition to one or more of the preceding or following aspects, or in the alternative to some aspects, the method can further include depositing a tie layer over the substrate prior to depositing the first hydrophilic layer.

In an eighty-first aspect, in addition to one or more of the preceding or following aspects, or in the alternative to some aspects, the substrate includes a metal or a polymer.

In an eighty-second aspect, in addition to one or more of the preceding or following aspects, or in the alternative to some aspects, the in-solution coating technique includes immersing the substrate in a solution of the photo-PVP compound at a concentration of less than 20 mg/ml and applying UV radiation while the substrate can be immersed.

In an eighty-third aspect, in addition to one or more of the preceding or following aspects, or in the alternative to some aspects, the concentration of the photo-PVP compound in the solution can be from 0.5 to 15 mg/ml.

In an eighty-fourth aspect, in addition to one or more of the preceding or following aspects, or in the alternative to some aspects, the heparin layer can be applied in multiple coats.

In an eighty-fifth aspect, in addition to one or more of the preceding or following aspects, or in the alternative to some aspects, depositing the heparin layer over the hydrophilic layer can be performed using a dip coating technique with a solution of the photo-heparin at a concentration of 50 to 100 mg/ml.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a coated medical device in accordance with various embodiments herein.

FIG. 2 is a cross-sectional view of a portion of the surface of a medical device as taken along line 2-2 of FIG. 1 in accordance with various embodiments herein.

FIG. 3 is a cross-sectional view of a portion of the surface of a medical device in accordance with various embodiments herein.

FIG. 4 is a cross-sectional view of a portion of the surface of a medical device in accordance with various embodiments herein.

FIG. 5 is a cross-sectional view of a portion of the surface of a medical device in accordance with various embodiments herein.

FIG. 6 is a cross-sectional view of a portion of the surface of a medical device in accordance with various embodiments herein.

FIG. 7 is a cross-sectional view of a portion of the surface of a medical device in accordance with various embodiments herein.

FIG. 8 is a cross-sectional view of a portion of the surface of a medical device in accordance with various embodiments herein.

FIG. 9 is a chart showing relative thrombus accumulation for various coatings made as described in the examples herein.

FIG. 10 is a chart showing fibrinogen absorption for various coatings made as described in the examples herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Coatings herein can exhibit thromboresistant and/or anti-fouling properties that are useful with various types of medical devices including, but not limited to, intravascular devices. Embodiments herein include multilayer thromboresistant coatings and medical devices including the same. While not intending to be bound by theory, certain coatings herein with anti-thrombogenic and/or antifouling properties can limit protein adhesion.

Various embodiments of coatings herein also exhibit surprising levels of lubricity. This characteristic can be beneficial for a wide variety of medical devices such as catheters, stents (including neurovascular stents), and other intravascular devices to reduce friction during insertion and operation. The lubricity of the coating can enhance patient comfort and reduce the risk of damage to blood vessels and other tissues.

Heparin is a compound that has long been used as an intravenous anticoagulant to treat inherent clotting disorders and to prevent blood clot formation during surgery and interventional procedures. Heparin molecules are polysaccharides with a unique chemical structure that gives them specific biological activity.

When heparin is immobilized onto the surface of a medical device material, it can improve the performance of the material when in contact with blood in several ways: 1) it can provide local catalytic activity to inhibit several enzymes critical to the formation of fibrin (which holds thrombi together); 2) it can reduce the adsorption of blood proteins, many of which lead to undesirable reactions on the device surface; and 3) it can reduce the adhesion and activation of platelets, which are a primary component of thrombus.

Heparin compounds can be modified to include a photoreactive group (photo-heparins) to facilitate their attachment to other components, without losing its bioactivity. Photo-heparins can be incorporated into a solution and applied to a substrate, such as by dip-coating or spray-coating. The coated substrate can then be irradiated to activate the photoreactive groups of the photo-heparin and bond the heparin molecule to the substrate and form a heparin-based hemocompatible coating. However, a limitation of heparin molecules is that relatively few groups that can be functionalized with photoreactive groups, which limits the overall photo-reactivity of photo-heparin. To deal with the low photo-reactivity of photo-heparin, one strategy is to apply it along with another photopolymer. However, that strategy may generate a thicker overall coating (>1000 nm), which is unsuitable for many intravascular applications. Further, any unbound photo-heparin in the coating can interfere with the properly bound photo-heparin and lower the overall activity of the coating. Alternatively, the UV irradiation time of the photo-heparin coating can be increased to ensure more photoreactive groups are activated. However, heparin can be susceptible to degradation from increased heat caused by the prolonged irradiation time, depending on the underlying substrate.

However, thromboresistant coatings herein can include one or more heparin compounds while desirably minimizing the thickness of the coating while maintaining or even improving hemocompatible activity of the hemocompatible coating.

In various embodiments, the thromboresistant coating can include a medical device with an antithrombogenic coating deposited on a substrate including an optional tie layer disposed over the substrate, a hydrophilic layer disposed over the tie layer, and a heparin layer disposed over the hydrophilic layer. The hydrophilic layer can include a PVP compound with a photo-activatable group (a photo-PVP compound) and the heparin layer can include a heparin compound with a photo-activatable group (a photo-heparin compound). The layers deposited on the substrate are typically remarkably thin. By way of example, the optional tie layer, the hydrophilic layer, and the heparin layer are typically less than or equal to 100 nm thick in total.

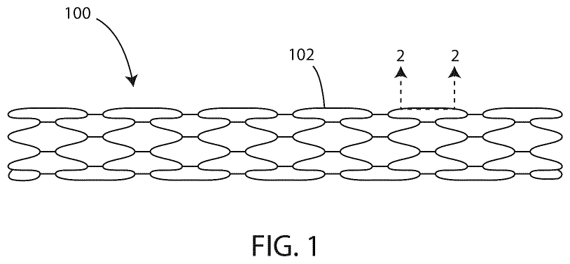

Referring now to FIG. 1, a schematic view of a coated medical device 100 is shown in accordance with various embodiments herein. In various embodiments, the medical device 100 can be a stent of various types. In various embodiments, the medical device 100 can be a neurovascular device, such as a neurovascular stent. However, many different types of medical devices are contemplated herein as described in greater detail below. In this example, the medical device 100 includes stent body 102, which can include struts, crowns, peaks, connectors, and the like. The stent body 102 and/or other portions of the medical device 100 can be coated with a coating as described herein.

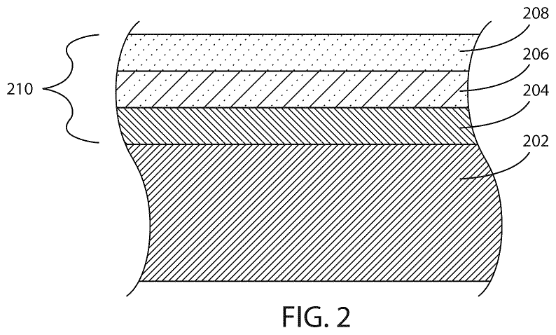

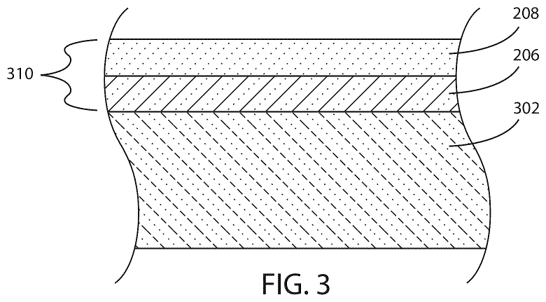

As shown in FIGS. 2-3, a hemocompatible coating, according to various embodiments of the present invention, can include a photo-heparin layer over a low-concentration photopolymer “primer” layer. The photopolymer primer layer can improve bonding of the photo-heparin to the substrate while maintaining or improving the photo-activity of the hemocompatible coating. The improved bonding of the photo-heparin to the substrate provided by the photopolymer primer layer allows for a lighter or thinner photo-heparin application, which reduces the overall thickness of the hemocompatible coating. In various embodiments, the hemocompatible coating can be between about 30 to 160 nm in thickness. In some embodiments, the hemocompatible coating can be about 40 to 90 nm in thickness.

The lighter or thinner photo-heparin application can reduce the heparin load of the hemocompatible coating, according to an embodiment of the present invention, relative to the higher heparin load of a thicker hemocompatible coating. However, it has been found that the thinner hemocompatible coatings herein can perform equivalently in reducing the formation of thrombus to the conventional heparin coating despite the lower heparin load. Further, photo-heparin containing coatings herein demonstrate equivalent heparin activity to the conventional heparin coating despite the substantially lower heparin load.

In an example, the low-concentration photopolymer layer or layers can include a hydrophilic polymer functionalized with at least one photoreactive group. While not intending to be bound by theory, the hydrophilic photopolymers can aid in the bonding of the photo-heparin to the substrate over a bare, unprimed substrate surface. Typically, hydrophilic photopolymers can be deemed unsuitable for hemocompatible and other long-term coatings as the attracted water can lead to swelling of the coating and corresponding degradation of the coating over an extended time in vivo, shedding particulates. The degradation of the hydrophilic polymers can weaken the overall coating causing portions of the entire coating, including the photo-heparin, to fragment from the coating. Further, certain hydrophilic polymers can cause inflammation when released from the coating due to degradation of the coating and absorption of the coating fragments into surrounding tissues. However, in various embodiments herein, the photopolymer is present in relatively low concentrations. The low concentration minimizes the water absorbed by the coating in vivo minimizing the swelling of the coating that can result in degradation. Similarly, the low concentration of photopolymer minimizes the risk that any degradation of the photopolymer will result in significant degradation of the coating as whole. Further, the low-concentration photopolymer layer can be positioned between the substrate and the photo-heparin layer, wherein the photo-heparin layer can act as a barrier limiting interaction between the photopolymer and the environment. This arrangement further limits perceived weaknesses associated with the use of hydrophilic photopolymers.

As shown in FIG. 2, the low-concentration photopolymer primer layer can be coated onto a substrate surface via a tie-layer basecoat. Certain substrates, such as metals, lack functional groups that can readily react with the photoreactive groups of the photopolymer to covalently bond the photopolymer directly to the substrate. A tie-layer basecoat, according to an embodiment of the present invention, can comprise a tie-layer reagent having a first functional group that bonds to the substrate and a second functional group that can react with the photopolymer. The tie-layer reagent can be bonded to the substrate surface and present the second functional group for reaction with the photoreactive groups of photopolymer. The tie-layer reagent can comprise a silane compound comprising bis(trimethoxysilyl)hexane, bis(trimethoxysilyl)ethane, bis(trimethoxysilylethyl)benzene, or a mixture thereof as disclosed in and made by the processes disclosed in U.S. Pat. No. 6,706,408, which is herein incorporated by reference.

Referring now to FIG. 2, a cross-sectional view is shown of a portion of the surface of a medical device as taken along line 2-2 of FIG. 1 in accordance with various embodiments herein. The surface includes a substrate 202, a tie layer 204, a thin hydrophilic layer 206, and an anti-thrombogenic active agent layer 208. The substrate 202 can be formed of various materials, such as metals, polymers, glasses, ceramics, and the like depending on the specific type of medical device. Examples of substrate materials are described in greater detail below. Depending on the nature of the substrate 202, a tie layer 204 can be included to aid in adhesion of coating layers onto the substrate 202. In some embodiments, the tie layer 204 can include one or more silane compounds. However, various other compounds are also contemplated herein. Examples of tie layer 204 components are described in greater detail below. The anti-thrombogenic active agent layer 208 can include various active agents with anti-thrombogenic compounds. By way of example, the anti-thrombogenic active agent can include a heparin compound. In some embodiments, the anti-thrombogenic active agent can include a photo-heparin compound (a heparin compound including a photo-reactive group). Other examples of anti-thrombogenic compounds such as phosphonates and the like are described in greater detail below.

The overall thickness 210 of the various layers can be remarkably thin. For example, the overall thickness 210 of the various layer can be less than about 200, 175, 150, 125, 100, 90, or 80 nm, or a thickness falling within a range between any of the foregoing.

One or more layers of the coating can be formed to be extremely thin using coating techniques including submerging a substrate to be coated into a dilute solution and applying UV radiation while the substrate is submerged (an in-solution coating technique). By way of example, in the context of FIG. 2, the thin hydrophilic layer 206 and/or the anti-thrombogenic active agent layer 208 can be formed using a dilute solution along with in-solution coating deposition. Other layers can be applied using conventional coating techniques including dip coating, spray coating, and the like.

In some embodiments, in-solution coating techniques can be used to apply multiple layers of a particular compound with each coat being extremely thin. For example, a first coat of an anti-thrombogenic active agent can be applied using an in-solution coating technique following by rinsing and then another coat or multiple coats can be applied. In some embodiments, the anti-thrombogenic active agent layer 208 can include 1, 2, 3, 4, 5, 6, 7, or 8 coats, or a number of coats falling within a range between any of the foregoing such as from 1 to 8 coats, from 2 to 6 coats, or from 2 to 4 coats.

In some embodiments the photo-PVP can include additional photogroups (by mixing in one or more photo-crosslinkers such as sodium bis(4-benzoylphenyl phosphate) or others described herein, or by adding one or more photo-crosslinkers on top of the photo-PVP via in-solution UV irradiation. Subsequently unmodified heparin, PSBMA or other polymers, without their own incorporated photogroups, can be linked on the surface by UV irradiation (in-solution, or after drying).

It will be appreciated that, in some embodiments, the tie layer can be omitted. As shown in FIG. 3, the low-concentration photopolymer primer layer can be coated directly onto a substrate surface. Certain substrate materials, such as polymers, can be directly reacted with photoreactive groups of the photopolymer to bond the photopolymer directly to the substrate surface.

Referring now to FIG. 3, a cross-sectional view is shown of a portion of the surface of a medical device in accordance with various embodiments herein. In this example, the surface includes a substrate 302, a thin hydrophilic layer 206, and an anti-thrombogenic active agent layer 208. The substrate 302 can be formed of a material that exhibits a desirable level of adhesion with other layers of a coating described herein without the need for a discrete tie layer.

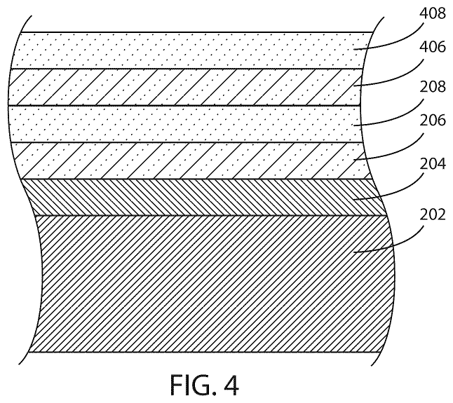

As shown in FIG. 4, in at least one embodiment, multiple hemocompatible coating coats can be applied. In this arrangement, an additional low-concentration photopolymer primer layer is applied over a photo-heparin top layer of a heparin coated substrate before an additional photo-heparin layer is applied over the additional low-concentration photopolymer primer layer. This process can be repeated to provide multiple alternating photo-heparin and photopolymer primer layers. This arrangement can efficiently increase the heparin load of the overall coating on the substrate while minimizing the unbounded photo-polymer. Thus, in some embodiments, alternating layers of different materials can be used herein. By way of example, alternating PVP and heparin layers can be used herein. Referring now to FIG. 4, a cross-sectional view is shown of a portion of the surface of a medical device in accordance with various embodiments herein. In this example, the medical device surface includes a substrate 202, a tie layer 204, a first thin hydrophilic layer 206, a first anti-thrombogenic active agent layer 208, a second thin hydrophilic layer 406, and a second anti-thrombogenic active agent layer 408. The overall thickness of the coating can still be quite thin. By way of example, the overall thickness of the coating can be less than 200, 175, 150, 125, or even 100 nm thick, or can fall within a range between any of the foregoing.

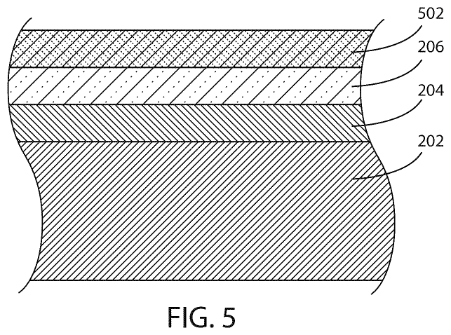

As shown in FIG. 5, in at least one embodiment, a photopolymer can be included in photo-heparin solution. The photopolymer in the photo-heparin solution can react with the photo-heparin and the photopolymer primer to further assist in the photo-heparin in bonding to the substrate. Thus, in some embodiments, an outer heparin layer can also include a photo-PVP compound disposed therein. Referring now to FIG. 5, a cross-sectional view is shown of a portion of the surface of a medical device in accordance with various embodiments herein. The device surface includes a substrate 202, a tie layer 204, a thin hydrophilic layer 206, and a combination PVP and heparin layer 502. The combination PVP and heparin layer 502 can include PVP compounds (such as photo-PVP compounds) and heparin compounds as described herein.

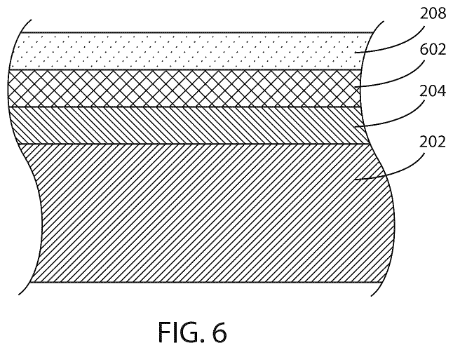

In some embodiments, other compounds can be included with one or more coating layers herein, such as iodine. Referring now to FIG. 6, a cross-sectional view is shown of a portion of the surface of a medical device in accordance with various embodiments herein. The surface includes a substrate 202, a tie layer 204, a combination PVP and iodine layer 602, and an anti-thrombogenic active agent layer 208. In various embodiments, the iodine can elute out of the medical device coating in the presence of bodily fluids, such as after the device is inserted or implanted into the body.

In some embodiments, the hemocompatible coating can further comprise a photopolymer topcoat over the photo-heparin layer. The photopolymer topcoat can act as a masking or protective layer for the heparin molecules. Heparin can inactivate thrombin molecules in the blood to prevent thrombosis formation near the substrate. However, fibrinogen molecules in the blood can bind to the bound heparin molecules, which can trigger platelet activation to release additional thrombin causing thrombosis formation despite the presence of heparin. The photopolymer topcoat prevents or restricts binding fibrinogen to the bound heparin molecules of the photo-heparin layer, which improves the overall effectiveness of the heparin coating.

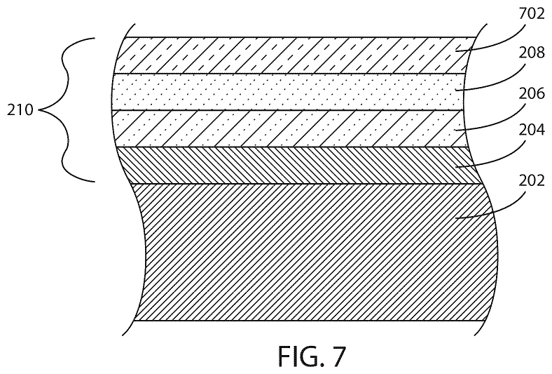

Referring now to FIG. 7, a cross-sectional view of a portion of the surface of a medical device is shown in accordance with various embodiments herein. As before, the surface includes a substrate 202, a tie layer 204, a thin hydrophilic layer 206, and an anti-thrombogenic active agent layer 208. However, in the embodiment of FIG. 7, a photo-PVP layer 702 is disposed over the other layers. The photo-PVP layer 702 is extremely thin and can be deposited by using an in-solution coating technique herein (submerging the device to be coated into a relatively dilute solution of a photo-PVP compound in a solvent and then applying actinic radiation, such as UV light, to the device while submerged). It has been surprisingly found that such thin photo-PVP compounds can provide remarkable enhancements of thromboresistant properties.

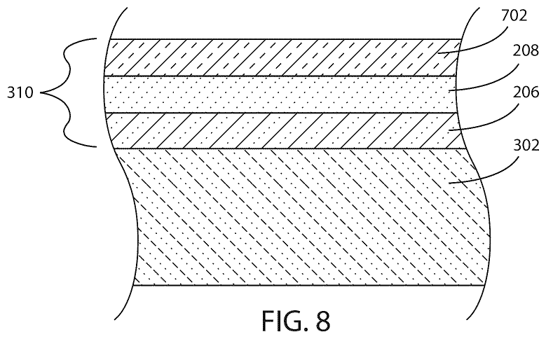

Referring now to FIG. 8, a cross-sectional view of a portion of the surface of a medical device is shown in accordance with various embodiments herein. FIG. 8 is generally similar to FIG. 7. However, in FIG. 8, the tie layer 204 is omitted. In FIG. 8, the surface of the medical device can still include a substrate 202, a thin hydrophilic layer 206, an anti-thrombogenic active agent layer 208, and a photo-PVP layer 702 disposed on top. As before, the photo-PVP layer 702 can be extremely thin and can be formed by using an in-solution coating technique.

### Heparin Compounds

Various embodiments herein include one or more heparin layers including one or more heparin compounds. Further details about exemplary heparin compounds are provided as follows. However, it will be appreciated that this is merely provided by way of example and that further variations are contemplated herein.

Various embodiments herein can include a heparin compound. Heparin is a glycosaminoglycan. Heparin compounds herein can include all molecular weights of heparin, low molecular weight heparin, heparan sulfate, heparan sulfate proteoglycans, sodium heparin, high affinity heparin, low affinity heparin, heparin fragments, heparin derivatives, and the like. Heparin compounds herein can include enoxaparin, dalteparin, tinzaparin, danaparoid.

Heparin compounds used herein can also include those modified to include a photoreactive group (“photoderivatized heparin” or “photo-heparin”). Photoderivatized heparin can be prepared by those skilled in the art, such as in the manner described in U.S. Pat. No. 5,563,056 (Swan et al., Preparation of Crosslinked Matrices Containing Covalently Immobilized Chemical Species and Unbound Releasable Chemical Species) and as described in U.S. Pat. No. 7,550,444, the contents of both of which are herein incorporated by reference. As one example, a photoreactive heparin (shown below, Compound I) can be prepared by reacting heparin with benzoyl-benzoyl-epsilon-aminocaproyl-N-oxysuccinimide in dimethylsulfoxide/carbonate buffer, pH 9.0. The solvent can be evaporated and the photoheparin dialyzed against water and lyophilized, and then dissolved in water at a desired concentration. The product can be referred to as BBA-EAC-heparin (referring to the benzophenone photoreactive group benzoyl benzoic acid (BBA) and the spacer, epsilon aminocaproic acid (EAC)). However, some embodiments herein can lack a heparin compound. In addition, other photo groups can be used beyond benzoyl benzoic acid (BBA). The photo-heparin can have a relatively low photo-load (referring to the ratio of subunits with photoreactive groups versus subunits without photoreactive groups-such as illustrated with respect to Compound I below) which desirably reduces crosslinking to itself. The ratio of subunits with photoreactive groups to subunits without photoreactive groups can be less than 1:1, 1:2, 1:5, 1:10, 1:20, 1:50, or 1:100, or less, or an amount falling within a range between any of the foregoing.

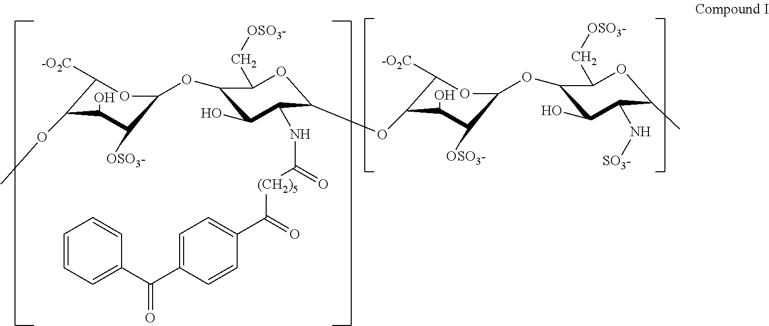

In some embodiments, a relatively thin layer of heparin (photo-heparin or non-photo heparin) can be applied in multiple coats with exposure to actinic radiation (such as UV light) and then rinsing in between coats.

In an example, the photo-heparin layer can be applied to the substrate primed with the photopolymer primer layer by immersing the substrate in a photo-heparin solution for a predetermined time. The photo-heparin solution can comprise photo-heparin dissolved in a solvent. In an example, the photo-heparin solution can comprise a concentration of between about 70 to 90 mg/ml photo-heparin in a 20% isopropyl alcohol solution. In another example, the photo-heparin solution can be further diluted in an organic solvent to a concentration of about 1 to 10 mg/ml photo-heparin in 20% isopropyl alcohol. In various embodiments, the solvent can include from 10 to 20% isopropyl alcohol. The lower concentration of the photo-heparin solution lowers the viscosity of the photo-heparin solution, which can cause filming and webbing of the photo-heparin on a substrate. Filming and webbing of the photo-heparin solution on the substrate can slow drying of the photo-heparin solution on the substrate, which can result in an uneven coating. The immersed substrate can be wet irradiated within the photo-heparin solution before being removed and dried. In at least one example, the photo-heparin solution coated substrate can be removed and dried without being irradiated. The photo-heparin solution can be dried by at least one of a low flow rate nitrogen gas stream, a high flow rate nitrogen gas stream between about 80 and 120 lpm, nitrogen chamber with a relative humidity of about 10%, vacuum dried with heat (about 50° C.), and wicking of the excess liquid photo-heparin solution from the substrate surface. The unirradiated photo-heparin coated substrate is then dry irradiated with UV radiation. In an embodiment, the substrate can be dry irradiated for about 15 to about 25 seconds. The substrate can be irradiated multiple times. In an embodiment, the substrate can be irradiated at least two cycles of about 15 to about 25 seconds and permitted to cool between each cycle. In some embodiments, after irradiation, the device is then rinsed and dried under heat. In some embodiment, the photo-heparin steps can be repeated at least twice to form multiple photo-heparin layers, however in some embodiments 1, 2, 3, 4, or more coats can be applied.

Various embodiments herein include a functional layer with heparin activity that may include one or more compounds that mimick heparin activity and may or may not include heparin or a heparin derivative. Heparin-mimicking compounds mimic the structure and function of naturally occurring heparan sulfate chains. Heparin-mimicking compounds can include, but are not limited to, heparin-mimicking small molecules, polymers, polypeptides, polysaccharides, and the like. Heparin-mimicking compounds herein can be modified to include a photoreactive group, such as a benzoyl benzoic acid (BBA) group.

Heparin-mimicking polymers are synthetic analogs designed to replicate the biological activities of heparin. Such polymers are engineered to mimic the unique structural features and biological functions of heparin, including its ability to interact with various proteins involved in the coagulation cascade and its capacity to inhibit the activity of certain enzymes, such as thrombin and factor Xa, which are key players in blood clot formation. Heparin-mimicking polymers can include, but are not limited to, modified dextrans, sulfated glycopolymers, polysulfonated compounds, sulfonated ionomers, and polyaromatic anionic compounds.

Heparin-mimicking polypeptides are polypeptides designed to replicate the biological functions of heparin and can include polypeptides synthesized to mimic the sulfated polysaccharide structures of heparin, enabling them to interact with various proteins involved in the coagulation cascade, such as antithrombin III, to inhibit thrombus formation. Some examples of such polypeptides can incorporate specific amino acid sequences that can be post-translationally modified to introduce sulfate or sulfonate groups, thereby mimicking the negative charge density and spatial configuration of heparin.

### Poly-Zwitterion Polymers and Copolymers

Polyzwitterionic polymers are generally defined as polymers having a backbone formed from a mixture of anionic and cationic monomers or blocks. These polymers often comprise a non-polymer and hydrophobic backbone with polar and hydrophilic side groups.

In various embodiments, polyzwitterion compounds can be used in a layer of a coating herein either in place of or in addition to a photo-heparin layer. For example, in some embodiments, an outermost layer of a coating herein can be formed of polyzwitterion compounds. Polyzwitterion compounds herein can include polymers or copolymers including both anionic and cationic groups thereon and, more specifically, any polymer or copolymer in which the monomers are zwitterions.

Polyzwitterion compounds herein can include, but are not limited to, homopolymers and copolymers including polyphosphobetaines, polysulfobetaines, polycarbobetaines, polyphosphocholines, polytrimethylamine N-oxide, polyectoine, poly(3-(N-(2-(methacryloyloxy)ethyl)-N,N-dimethylammonio) propanesulfonate), poly-N-(carboxymethyl)-N,N-dimethyl-2-(methacryloyloxy) ethanaminium) (PCDME).

Polyzwitterion compounds herein can specifically include zwitterionic poly(sulfobetaine methacrylate) (PSBMA). In some embodiments, polyzwitterionic compounds herein can include those with photoreactive groups (including but not limited to benzophenone photoreactive groups).

### Hydrophilic Photopolymers

In at least one embodiment, the photopolymer can comprise polymers may include several distinct polymer types, as prepared by terminal or side chain grafting. The polymers of the invention may include cellulose-based products such as hydroxyethyl cellulose, hydroxypropyl cellulose, carboxymethyl cellulose, cellulose acetate and cellulose butyrate, acrylics such as those polymerized from hydroxyethyl acrylate, hydroxyethyl methacrylate, glyceryl acrylate, glyceryl methacrylate, acrylic acid, methacrylic acid, acrylamide and methacrylamide, vinyls such as polyvinyl pyrrolidone and polyvinyl alcohol, nylons such as polycaprolactam, polylauryl lactam, polyhexamethylene adipamide and polyhexamethylene dodecanediamide, polyurethanes, polylactic acids, linear polysaccharides such as amylose, dextran, chitosan, and hyaluronic acid, and branched polysaccharides such as amylopectin, hyaluronic acid and hemi-celluloses. Suitable polymerizable compounds can also comprise electrically neutral hydrophilic functional units, for example, acrylamide and methacrylamide derivatives. Examples of suitable monomers containing electrically neutral hydrophilic structural units include acrylamide, methacrylamide, N-alkylacrylamides (e.g., N,N-dimethylacrylamide or methacrylamide, N-vinylpyrrolidinone, N-vinylacetamide, N-vinyl formamide, hydroxyethylacrylate, hydroxyethylmethacrylate, hydroxypropyl acrylate or methacrylate, glycerolmonomethacrylate, and glycerolmonoacrylate). The photoreactive groups of the photopolymer can be provided in the form of a heat activatable group (e.g., an azide group). As used herein, a photoreactive group is capable of being activated to form a covalent bond via hydrogen abstraction upon illumination with light of the appropriate wavelength. Such photoreactive groups are preferably also capable, if unable to abstract a hydrogen, of reverting to an inactive, or “latent reactive,” state. Thus, upon illumination with light of a suitable wavelength, a first photoreactive group of the photopolymer can be those that covalently bind to the support surface by abstracting a hydrogen from the surface. A second photoreactive group, on the other hand, are those that remain unreacted and thereafter revert to a latent reactive state. The second photoreactive group thereby remains available for initiating polymerization. When the first and second photoreactive groups are different, they may, in a preferred embodiment, be activatable by light of different wavelengths, such that light of a particular wavelength will activate the first photoreactive groups but not the second, and vice versa. The photopolymer can comprise those disclosed in and made by the processes disclosed in U.S. Pat. Nos. 4,973,493; 4,979,959; 5,217,492; 5,002,582; 5,741,551; and 6,669,994, which are herein incorporated by reference.

In an example, the low-concentration photopolymer primer layer can be coated onto the substrate by immersing the bare substrate or the tie-layer coated substrate in a low-concentration photopolymer solution and applying UV radiation while the substrate is submerged (an in-solution coating technique). The low-concentration photopolymer can comprise photopolymer dissolved in a solvent. The photopolymer solution can include about 0.5 to 10 mg/ml of the photo polymer. In some embodiments, the photopolymer solution can include about 0.1, 0.5, 1, 2, 3, 4, 5, 6, 7, 8 mg/ml or more of the photo polymer, or an amount falling within a range between any of the foregoing. The solvent can include a mixture of different components. In some embodiments, the solvent can include a mixture of isopropyl alcohol and water. In some embodiments, a solvent herein can include about 10, 15, 20, 25, 30 percent isopropyl alcohol (volume basis), or an amount falling within a range between any of the foregoing, with the remainder being water. It will be appreciated, however, that other solvent formulations are also contemplated herein.

The substrate can be “wet irradiated” while immersed within the low-concentration photopolymer solution for about 15 to about 45 seconds. In at least one embodiment, the substrate can be irradiated at least two cycles of about 15 to 24 seconds and allowed to cool after each cycle. Certain substrates, such as metal substrates, can become heated during irradiation, which can affect the photopolymer. The cooling cycle allows the photopolymer and the photopolymer solution to cool between irradiation steps to minimize any impact on the photopolymer. The low-concentration photopolymer steps can be repeated to form multiple low-concentration photopolymer layers on the substrate.

### Polyvinylpyrrolidone Polymers

Various embodiments herein include a polyvinylpyrrolidone (PVP) polymer, such as in a PVP layer. Further details about the polyvinylpyrrolidone are provided as follows. However, it will be appreciated that this is merely provided by way of example and that further variations are contemplated herein.

Polyvinylpyrrolidone polymers herein can include polyvinylpyrrolidone homopolymers as well as polyvinylpyrrolidone subunit containing copolymers. By way of example, polyvinylpyrrolidone copolymers can include subunits of polyvinylpyrrolidone as follows:

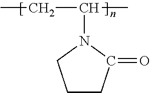

Polyvinylpyrrolidone polymers herein can be linear or can be branched. Polyvinylpyrrolidone polymers herein can have various molecular weights such as an average molecular weight from 1 kDa to 3000 kDa. In various embodiments, the polyvinylpyrrolidone polymer has an average molecular weight from 10 kDa to 50 kDa. In various embodiments, a non-photoreactive polyvinylpyrrolidone is a blend of different molecular weight PVP compounds. Exemplary non-photo derivatized polyvinylpyrrolidone polymers can include, for example, PVP K12, PVP K30, PVP K90, and the like. Polyvinylpyrrolidone polymers herein can be used to form a PVP hydrogel.

In various embodiments, the polyvinylpyrrolidone can include a non-photoreactive polyvinylpyrrolidone. However, instead of or in addition to non-photoreactive polyvinylpyrrolidone, in various embodiments, the polyvinylpyrrolidone can also include a photoreactive polyvinylpyrrolidone (photo-PVP). Photoreactive polyvinylpyrrolidones can serve as a hydrophilic photopolymer herein. Photoreactive polyvinylpyrrolidones can include homopolymers and/or copolymers where they are derivatized to include a photoreactive group. In various embodiments, the photoreactive polyvinylpyrrolidone can specifically include a benzophenone group.

An exemplary photoreactive polyvinylpyrrolidone copolymer can include poly[vinyl pyrrolidone-co-N-(3-(4-benzoylbenzamideo)propyl)methacrylamide] (or PVP-co-APMA with 80 to 99.9 mole percent PVP and 20 to 0.1 mole percent APMA). By way of example, an exemplary photoreactive polyvinylpyrrolidone is as follows:

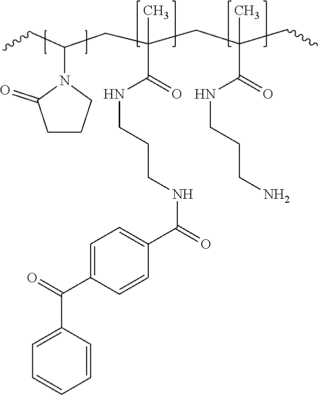

In some embodiments, the PVP-co-APMA can include approximately 99 mole % N-vinylpyrrolidone and 1 mole % APMA.

Another exemplary photoreactive polyvinylpyrrolidone copolymer (acetylated PVP-APMA-BBA; or acetylated photo-PVP) is as follows:

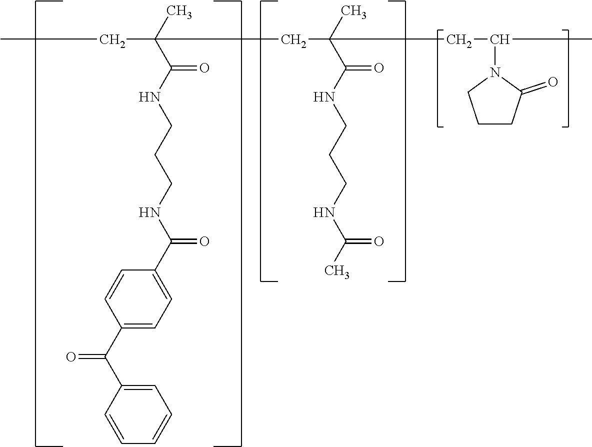

This compound can be prepared by a copolymerization of 1-vinyl-2-pyrrolidone and N-(3-aminopropyl)methacrylamide (APMA, followed by photoderivatization of the polymer using 4-benzoylbenzoyl chloride under Schotten-Baumann conditions. The unreacted amines of the photopolymer can be further acetylated using acetic anhydride.

In various embodiments, photo reactive PVP compounds herein can be formed as described in U.S. Pat. Nos. 4,973,493; 4,979,959; 5,002,582; 5,217,492; 5,263,992; 5,512,329; 5,741,551; 6,669,994; and 7,087,658, the content of which is herein incorporated by reference.

The PVP layer can be extremely thin. In some embodiments, the PVP layer can be applied from a solution at a relatively low concentration. By way of example, the PVP layer can be applied by forming a relatively low concentration solution of a photoreactive PVP compound in a solvent, such as from 0.5 to 10 mg/ml, 2 to 8 mg/ml, 5 to 7 mg/ml, or 6 mg/ml and submerging the device to be coated into the solution and then applying actinic radiation, such as UV light, to the device while submerged. In some embodiments, the device can then be rinsed. In some embodiments, the process can be repeated.

### Polyacrylamide Polymers

Various embodiments herein include a polyacrylamide (PA) polymer or copolymer. Further details about polyacrylamide polymers are provided as follows. However, it will be appreciated that this is merely provided by way of example and that further variations are contemplated herein.

In some embodiments, the polyacrylamide polymer can be modified to include a photoreactive group, such as a BBA group or another photoreactive group. Photoreactive polyacrylamides can serve as a hydrophilic photopolymer herein.

In some embodiments, polyacrylamide polymers herein can include a photo-polyacrylamide-co-methacrylamide and/or a photo-polyacrylamide-co-methacrylate. Such compound can be prepared as described in U.S. Pat. Nos. 5,002,582 and 5,512,329, the content of which is herein incorporated by reference.

In some embodiments, the polymer comprising polyacrylamide can include acrylamido-2-methylpropanesulfonate (AMPS) segments. By way of example, poly[acrylamide-co-sodium-2-acrylamido-2-methylpropanesulfonate-co-N-(3-(4-benzoylbenzamido)propyl)methacrylamide] (PA-AMPS-BBA-MA) can be included and can be prepared according to the procedure described in U.S. Pat. Nos. 4,973,493, 5,002,582, and 5,263,992.

In some embodiments, the polymer comprising polyacrylamide can include acrylamido-2-methylpropanesulfonate groups (AMPS) and polyethyleneglycol segments. In a specific embodiment, the polymer comprising polyacrylamide can be N-Acetylated poly[acrylamide-co-sodium-2-acrylamido-2-methylpropanesulfonate-co-N-(3-(4-benzoylbenzamido)propyl)methacrylamide]-co-methoxy poly(ethylene glycol) monomethacrylate. Polymers comprising polyacrylamide in accordance with embodiments herein are described in U.S. Pat. Nos. 4,979,959; 5,263,992; and 5,512,329, the content of all of which is herein incorporated by reference in its entirety.

By way of example, the polyacrylamide compound can be N-Acetylated poly[acrylamide^{93.6%}-co-sodium-2-acrylamido-2-methylpropanesulfonate^{4.9%}-co-N-(3-(4-benzoylbenzamido)propyl)methacrylamide^{0.9%}]-co-methoxy poly(ethylene glycol)₁₀₀₀ monomethacrylate^{0.6%} (percentages are mole percents) (PA-BBA-AMPS-PEG). Reagents and methods for the preparation of PA-BBA-AMPS-PEG can be found in references such as U.S. Pat. Nos. 4,979,959; 5,002,582; 5,263,992; 5,414,075; 5,512,329; and 5,637,460, the content of all of which are incorporated herein by reference.

### Tie Layers

In some instances, a tie layer can be used with embodiments herein. In an example, a tie-layer basecoat can be coated onto the substrate by immersing the substrate in a tie-layer solution comprising a tie-layer reagent dissolved in a solvent. The tie-layer reagent comprises about 5 vol % of the tie-layer solution. In at least one embodiment, the tie-layer reagent can comprise about 1 to 10 vol % of the tie-layer solution. The substrates can be immersed in the tie-layer solution for about 60 minutes. In at least one embodiment, the substrate can be immersed between about 30 to 90 minutes. The tie-layer coated substrates can be removed from the tie-layer solution, washed with a solvent, and dried before being baked to set the tie-layer basecoat onto the substrate surface.

Exemplary tie layers include, but are not limited to those with silane compounds. Silane tie layers are described in US Patent Publication 2012/0148852 (to Jelle, et al.), the content of which is herein incorporated by reference.

The tie layer is attached to the substrate and includes a silane compound, a hydrolysis reaction product of the silane compound, a polymeric reaction product formed from the hydrolysis reaction product of the silane compound, or a combination thereof. The silane compound, a hydrolysis reaction product of the silane compound, a polymeric reaction product formed from the hydrolysis reaction product, or a combination thereof can bind to the surface of the inorganic substrate by reacting with oxide or hydroxide groups on the surface of the inorganic substrate. A covalent bond forms between the inorganic substrate and at least one compound in the first coating layer.

In some embodiments, the inorganic substrate can be treated to generate hydroxide or oxide groups on the surface. For example, the substrate can be treated with a plasma treatment or with a strong base such as sodium hydroxide, ammonium hydroxide, and the like. In the case of a metal, the metal can be subjected to an oxidizing potential to generate oxide or hydroxide sites on the surface of the metal. In some embodiments, a metal surface can be treated with hydrogen peroxide or other peroxides.

In some embodiments, at least some alkoxysilyl groups will undergo hydrolysis. The hydrolysis reaction product of the silane compound can typically polymerize to form a polymeric reaction product. Trimethoxysilyl groups usually undergo hydrolysis and subsequent polymerization more rapidly than either triethoxysilyl or tripropoxysilyl groups. A layer of the resulting polymeric material typically covalently binds to the surface of the inorganic substrate.

The silane compound has at least two tri(C1-C3)alkoxysilyl groups. Suitable groups include trimethoxysilyl, triethoxysilyl, and tripropoxysilyl, and combinations thereof. In some embodiments, the silane compound has at least two trimethoxysilyl groups. The silane is free of other groups that can bind to the inorganic substrate such as a sulfide group.

Examples of suitable tri(C1-C3)alkoxysilyl containing silane compounds include, but are not limited to, bis(trimethoxysilyl)hexane, bis(trimethyoxysilyl)ethane, and bis(trimethoxysilylethyl)benzene. A mixture of the tri(C1-C3)alkoxysilyl silane compounds can be used. In an embodiment, the silane compound is 1,4-bis(trimethoxysilylethyl)benzene. 1,4-bis(trimethoxysilylethyl)benzene can be prepared according to the procedures described in U.S. Pat. No. 6,706,408. In an embodiment, the silane compound is selected from those capable of forming hydrolytically stable siloxane bonds to the substrate.

Other types of compounds are contemplated herein for use as a tie layer. For example, phosphonate compounds including phosphonic acids, can be used as a tie layer. Examples of phosphonate compounds useful for tie layers can be found in U.S. Publ. Pat. Appl. No. 2023/0323152, the content of which is herein incorporated by reference.

### Substrates

The substrate can be formed from any desirable material, or combination of materials, suitable for use within the body. In some embodiments the substrate is formed from a metal such as various biocompatible metals including alloys. Exemplary metals can include, but are not limited to titanium, stainless steel, nitinol, various cobalt-chromium alloys, and the like. In some embodiments, the substrate is formed from a polymer. Polymers can include thermoplastics, thermosets, and elastomers. Specific polymers can include, but are not limited to acrylonitrile butadiene styrene (ABS0, polypropylene, polycarbonate, polyethylene, polyvinyl chloride, polyamide, polyetheretherketone (PEEK), various acrylics, and the like. In some embodiments, the substrate is formed from a glass or a ceramic material, such as alumina, zirconia, silicon nitride, and the like. In some embodiments, the substrate is formed from a composite material.

### Cross-Linking Agents

Various embodiments herein include a cross-linking agent. By way of example, in some embodiments, one or more cross-linking agents with photoreactive groups can be disposed within or on a hydrophilic layer herein and can be used to bond a heparin layer over the hydrophilic layer. This can allow binding of heparin, heparin derivatives, or heparin mimics that don't have photogroups themselves (e.g., are not photoreactive).

Further details about the cross-linking agent are provided as follows. However, it will be appreciated that this is merely provided by way of example and that further variations are contemplated herein. Further exemplary cross-linking agents are described in U.S. Publ. Pat. App. No. 2011/0245367, the content of which is herein incorporated by reference in its entirety.

In some embodiments, the crosslinking agent(s) can have a molecular weight of less than about 1500 kDa, but in other embodiments can be larger. In some embodiments the crosslinking agent can have a molecular weight of less than about 1200, 1100, 1000, 900, 800, 700, 600, 500, or 400 or less, or a molecular weight falling within a range between any of the foregoing.

In various embodiments, cross-linking agents include one or more photoreactive groups attached to a linking group. The cross-linking agent (or linking agent) can be represented by the formula Photo¹-LG-Photo², wherein Photo¹ and Photo² independently represent at least one photoreactive group and LG represents a linking group. The term “linking group” as used herein, refers to a segment or group of molecules configured to connect two or more molecule to each another. In some embodiments, the linking group can include a heteroatom. In some embodiments, the linking group lacks a heteroatom. In one embodiment, the linking group includes at least one silicon atom. In another embodiment, the linking group includes at least one phosphorus atom.

In some embodiments, the linking group can be a degradable linking group, which in other embodiments the linking group can be a non-degradable linking group. The term “degradable linking group” as used herein, refers to a moiety configured to connect one molecule to another, wherein the linking group is capable of cleavage under one or more conditions. The term “biodegradable” as used herein, refers to degradation in a biological system, and includes for example, enzymatic degradation or hydrolysis. It should be noted that the term “degradable” as used herein includes both enzymatic and non-enzymatic (or chemical) degradation. It is also understood that hydrolysis can occur in the presence of or without an acid or base. In one embodiment, the linking agent is water soluble. In another embodiment, the linking agent is not water soluble.

In various embodiments the linking group can function as a spacer, for example, to increase the distance between the photoreactive groups of the linking agent. For example, in some instances it may be desirable to provide a spacer to reduce steric hindrance that may result between the photoreactive groups, which could interfere with the ability of the photoreactive groups to form covalent bonds with a support surface, or from serving as a photoinitiator for polymerization. As described herein, it is possible to vary the distance between the photoreactive groups, for example, by increasing or decreasing the spacing between one or more photoreactive groups.

As described herein, one or more photoreactive groups can be bound to a linking group by a degradable or a non-degradable linkage. In various embodiments, the degradable linkage between the photoreactive group and the linking group includes at least one heteroatom, including, but not limited to oxygen, nitrogen, selenium, sulfur or a combination thereof. In one embodiment, a photoreactive group, linking group and heteroatom form an ether (R¹—O—R²), wherein R¹ is a photoreactive group and R² is a linking group. In another embodiment, a photoreactive group, linking group and heteroatom form an amine,

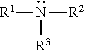

wherein R¹ is a photoreactive group, R² is a linking group, and R³ is hydrogen, aryl or alkyl, a photoreactive group, or a hydroxyl or salt thereof. In one embodiment, R³ is cyclic, linear or branched, saturated or unsaturated, aromatic or heteroaromatic, or a combination thereof. The stability of the ether and/or amine linkage can be influenced depending upon the size (e.g., chain length, branching, bulk, etc.) of the substituents. For example, bulkier substituents will generally result in a more stable linkage (i.e., a linking agent that is slower to degrade in the presence of water and/or acid).

In various embodiments, the linking group includes one or more silicon atoms. In a particular embodiment, the linking group includes one silicon atom (which can be referred to as a monosilane) covalently bound to at least two photoreactive groups. In another embodiment, the linking group includes at least two silicon atoms (which can be referred to as a disilane). In one embodiment, the linking group can be represented by the formula Si—Y—Si, wherein Y represents a linker that can be null (e.g., the linking group includes a direct Si—Si bond), an amine, ether, linear or branched C₁-C₁₀ alkyl, or a combination thereof. In one embodiment, Y is selected from O, CH₂, OCH₂CH₂O and O(CH₂CH₂O)ₙ, wherein n is an integer between 1 and 5, between 1 and 10, between 1 and 15, between 1 and 20, between 1 and 25, or between 1 and 30. One embodiment of a disilane linking agent is shown below

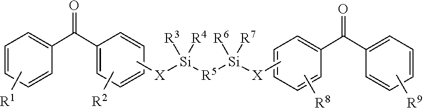

wherein R¹, R², R⁸ and R⁹ can be any substitution, including, but not limited to H, alkyl, halide, hydroxyl, amine, or a combination thereof; R³, R⁴, R⁶ and R⁷ can be alkyl, aryl or a combination thereof; R⁵ can be any substitution, including but not limited to O, alkyl or a combination thereof; and each X, independently, can be O, N, Se, S, or alkyl, or a combination thereof. One specific embodiment is shown below:

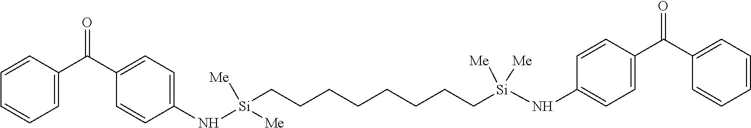 [SMILES: C[Si](C)(CCCCCCCC[Si](C)(C)Nc1ccc(C(=O)c2ccccc2)cc1)Nc1ccc(C(=O)c2ccccc2)cc1]

In various embodiments, the linking agent can be represented by the formula

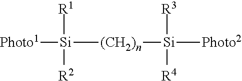

wherein Photo¹ and Photo², independently, represent one or more photoreactive groups and n is an integer between 1 and 10, wherein the linking agent comprises a covalent linkage between at least one photoreactive group and the linking group, wherein the covalent linkage between at least one photoreactive group and the linking group is interrupted by at least one heteroatom. In general, a longer hydrocarbon chain between the two silicon atoms will tend to increase the flexibility of the linking agent and may facilitate crosslinking between a greater number of polymers than a linking agent with a shorter carbon chain, since the photoreactive groups can react with polymers located farther apart from one another. In the formula shown above, R¹, R², R³, R⁴ are independently alkyl or aryl, including, but not limited to cyclic, linear or branched, saturated or unsaturated, aromatic or heteroaromatic, or a combination thereof. In a more particular embodiment, R¹-R⁴ are independently phenyl, methyl, ethyl, isopropyl, t-butyl, or a combination thereof. In another embodiment, R¹-R⁴ can also be, independently, a photoreactive group. In yet another embodiment, R¹-R⁴ can also be, independently, hydroxyl or salt thereof. In one embodiment, the hydroxyl salt includes a counterion that is lithium, sodium, potassium, or a combination thereof.

In another embodiment, the linking agent can be represented by the formula

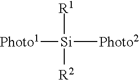

wherein Photo¹ and Photo², independently, represent one or more photoreactive group, wherein the linking agent comprises a covalent linkage between at least one photoreactive group and the linking group, wherein the covalent linkage between at least one photoreactive group and the linking group is interrupted by at least one heteroatom; R¹ and R² are independently alkyl or aryl, including, but not limited to cyclic, linear or branched, saturated or unsaturated, aromatic or heteroaromatic, or a combination thereof. In a more particular embodiment, R¹ and R² are independently phenyl, methyl, ethyl, isopropyl, t-butyl, or a combination thereof. R¹ and R² can also be, independently, a photoreactive group, wherein the linking agent comprises a covalent linkage between at least one photoreactive group and the linking group, wherein the covalent linkage between at least one photoreactive group and the linking group is interrupted by at least one heteroatom; or hydroxyl or salt thereof. In one embodiment, the hydroxyl salt includes a counterion that is lithium, sodium, potassium, or a combination thereof. One embodiment of a monosilane linking agent is shown below

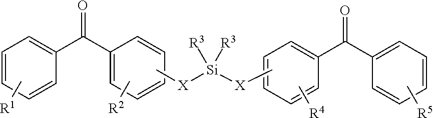

in which R¹ and R⁵ can be any substitution, including, but not limited to H, halogen, amine, hydroxyl, alkyl, or a combination thereof; R² and R⁴ can be any substitution, except OH, including, but not limited to H, alkyl or a combination thereof; R³ can be alkyl, aryl or a combination thereof, including, for example, methyl, ethyl, propyl, isopropyl and butyl; and X, independently, can be O, N, Se, S, alkyl or a combination thereof.

In another embodiment, the linking group includes one or more phosphorous atoms. In one embodiment, the linking group includes one phosphorus atom (which can also be referred to as a mono-phosphorus linking group). In another embodiment, the linking agent includes two phosphorus atoms (which can also be referred to as a bis-phosphorus linking group). In one embodiment, the linking group comprises at least one phosphorus atom with a phosphorus-oxygen double bond (P═O), wherein at least one or two photoreactive groups are bound to the phosphorus atom. In another embodiment, the linking group comprises one phosphorus atom with a phosphorus-oxygen double bond (P═O), wherein two or three photoreactive groups are covalently bound to the phosphorus atom. In another embodiment, the linking group comprises at least two phosphorus atoms, wherein at least one phosphorus atom includes a phosphorus-oxygen double bond (P═O), and at least one or two photoreactive groups are covalently bound to each phosphorus atom.

In a more particular embodiment, the linking agent can be represented by the formula:

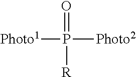

wherein Photo¹ and Photo², independently, represent one or more photoreactive groups, wherein the linking agent comprises a covalent linkage between at least one photoreactive group and the linking group, wherein the covalent linkage between at least one photoreactive group and the linking group is interrupted by at least one heteroatom and R is alkyl or aryl, a photoreactive group, hydroxyl or salt thereof, or a combination thereof. In one embodiment, the hydroxyl salt includes a counterion that is lithium, sodium, potassium, or a combination thereof. In a more particular embodiment, R is cyclic, linear or branched, saturated or unsaturated, aromatic or heteroaromatic, or a combination thereof. In a more particular embodiment, R is phenyl, methyl, ethyl, isopropyl, t-butyl, or a combination thereof.

In another embodiment, the linking agent can be represented by formula:

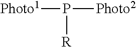

wherein Photo¹ and Photo² independently, represent one or more photoreactive groups, wherein the linking agent comprises a covalent linkage between at least one photoreactive group and the linking group, wherein the covalent linkage between at least one photoreactive group and the linking group is interrupted by at least one heteroatom and R is alkyl or aryl, a photoreactive group (wherein the covalent linkage between the photoreactive group and the linking group may be interrupted by at least one heteroatom), hydroxyl or salt thereof, or a combination thereof. In one embodiment, the hydroxyl salt includes a counterion that is lithium, sodium, potassium, or a combination thereof. In a more particular embodiment, R is cyclic, linear or branched, saturated or unsaturated, aromatic or heteroaromatic, or a combination thereof. In one embodiment, R is phenyl, methyl, ethyl, isopropyl, t-butyl, or a combination thereof.

In another embodiment, the linking agent can be represented by the formula:

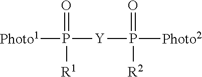

wherein Photo¹ and Photo², independently, represent one or more photoreactive groups, wherein the linking agent comprises a covalent linkage between at least one photoreactive group and the linking group, wherein the covalent linkage between at least one photoreactive group and the linking group is interrupted by at least one heteroatom; Y represents a linker that can be null (i.e., not present, such that the linking group includes a direct P—P bond), N or O, linear or branched C₁-C₁₀ alkyl, or a combination thereof, and R¹ and R² are independently alkyl, aryl, a photoreactive group (wherein the covalent linkage between the photoreactive group and the linking group can be interrupted by at least one heteroatom), hydroxyl or salt thereof, or a combination thereof. In one embodiment, Y is selected from O, CH₂, OCH₂O, OCH₂CH₂O and O(CH₂CH₂O)ₙ, wherein n is an integer between 1 and 5, between 1 and 10, between 1 and 15, between 1 and 20, between 1 and 25, or between 1 and 30. In one embodiment, the hydroxyl salt counterion is lithium, sodium, potassium, or a combination thereof. In a more particular embodiment, R¹ and R² are independently, cyclic, linear or branched hydrocarbon, saturated or unsaturated, aromatic or heteroaromatic, or a combination thereof. In one embodiment, R¹ and R² are independently phenyl, methyl, ethyl, isopropyl, t-butyl, or a combination thereof. In general, a longer hydrocarbon chain between the two phosphorus atoms will tend to increase the flexibility of the linking agent and may facilitate crosslinking between a greater number of polymers than a linking agent with a shorter carbon chain, since the reactive photoreactive groups can react with polymers located farther apart from one another. In one embodiment, Y can be O, CH₂, OCH₂CH₂O and O(CH₂CH₂O)ₙ wherein n is an integer between 1 and 5, between 1 and 10, between 1 and 15, between 1 and 20, between 1 and 25, or between 1 and 30. One embodiment is shown below

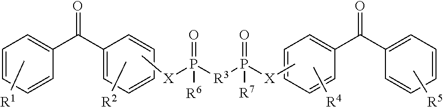

in which R¹, R², R⁴ and R⁵ can be any substitution, including but not limited to H, alkyl, halogen, amine, hydroxyl, or a combination thereof; R³ can be any substitution, including but not limited to O, alkyl, or a combination thereof; and each X can independently be O, N. Se, S, alkyl, or a combination thereof. In one embodiment, the linking agent includes one or more phosphorester bonds and one or more phosphoramide bonds, and can be represented by the formula:

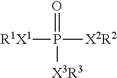

wherein X and X² are, independently, O, N, Se, S or alkyl; R¹ and R² are independently, one or more photoreactive groups, and X³ is O, N, Se, S, alkyl or aryl; R³ is alkyl or aryl, including, but not limited to cyclic, linear or branched, saturated or unsaturated, aromatic or heteroaromatic, or a combination thereof. In a more particular embodiment, R³ is phenyl, methyl, ethyl, isopropyl, t-butyl, or a combination thereof. R³ can also be a photoreactive group or a hydroxyl or salt thereof. In one embodiment, the hydroxyl salt counterion is lithium, sodium, potassium, or a combination thereof.

In one embodiment, the linking agent comprises a triphosphorester, which can be represented by the formula.

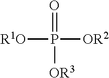

wherein R¹ and R² are independently, one or more photoreactive groups, and R³ is alkyl or aryl, including, but not limited to cyclic, linear or branched, saturated or unsaturated, aromatic or heteroaromatic, or a combination thereof. In a more particular embodiment, R³ is phenyl, methyl, ethyl, isopropyl, t-butyl, or a combination thereof. R³ can also be a photoreactive group or hydrogen, or a hydroxyl salt. In one embodiment, the hydroxyl salt counterion is lithium, sodium, potassium, or a combination thereof.

Some specific embodiments include the following linking agents:
(a) bis(4-benzoylphenyl) hydrogen phosphate:

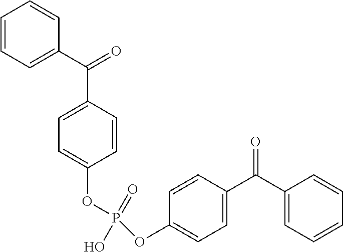 [SMILES: O=C(c1ccccc1)c1ccc(OP(=O)(O)Oc2ccc(C(=O)c3ccccc3)cc2)cc1]

(b) sodium bis(4-benzoylphenyl phosphate):

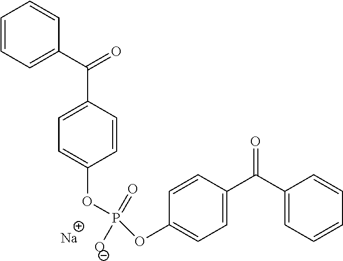 [SMILES: O=C(c1ccccc1)c1ccc(OP(=O)([O-])Oc2ccc(C(=O)c3ccccc3)cc2)cc1 ; [Na+]]

(c) tris(4-benzyolphenyl) phosphate):

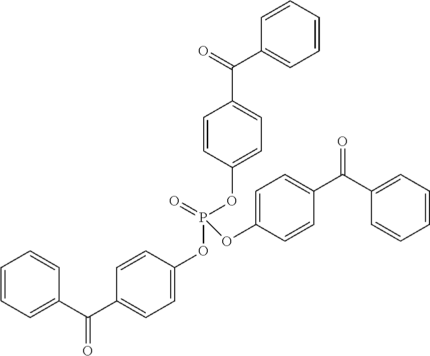 [SMILES: O=C(c1ccccc1)c1ccc(OP(=O)(Oc2ccc(C(=O)c3ccccc3)cc2)Oc2ccc(C(=O)c3ccccc3)cc2)cc1]

(d) tetrakis(4-benzoylphenyl)methylenebis(phosphonate)

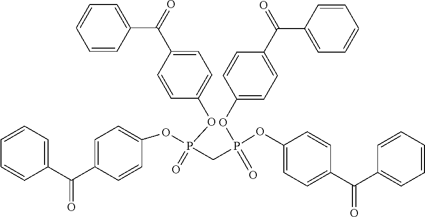 [SMILES: O=C(c1ccccc1)c1ccc(OP(=O)(CP(=O)(Oc2ccc(C(=O)c3ccccc3)cc2)Oc2ccc(C(=O)c3ccccc3)cc2)Oc2ccc(C(=O)c3ccccc3)cc2)cc1]

In another embodiment, the linking agent comprises a triphosphoramide, which can be represented by the formula.

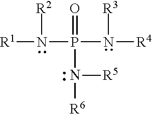

wherein R¹-R⁶ are independently, a photoreactive group, a hydroxyl or salt thereof, alkyl or aryl, or a combination thereof, wherein at least two of R¹-R⁶ are, independently, a photoreactive group. In one embodiment, the hydroxyl salt counterion is lithium, sodium, potassium, or a combination thereof. In a more particular embodiment, R¹-R⁶ are independently cyclic, linear or branched, saturated or unsaturated, aromatic or heteroaromatic, or a combination thereof. In a more particular embodiment, R¹-R⁶ are, independently, phenyl, methyl, ethyl, isopropyl, t-butyl, or a combination thereof.

In some embodiments, the photoactivatable cross-linking agent can be ionic, and can have good solubility in an aqueous composition, such as the first and/or second coating composition. Thus, in some embodiments, at least one ionic photoactivatable cross-linking agent is used to form the coating. In some cases, an ionic photoactivatable cross-linking agent can crosslink the polymers within the second coating layer which can also improve the durability of the coating.

Any suitable ionic photoactivatable cross-linking agent can be used. In some embodiments, the ionic photoactivatable cross-linking agent is a compound of formula I: X₁—Y—X₂ where Y is a radical containing at least one acidic group, basic group, or a salt of an acidic group or basic group. X₁ and X₂ are each independently a radical containing a latent photoreactive group. The photoreactive groups can be the same as those described herein. Spacers can also be part of X₁ or X₂ along with the latent photoreactive group. In some embodiments, the latent photoreactive group includes an aryl ketone or a quinone.

The radical Y in formula I provides the desired water solubility for the ionic photoactivatable cross-linking agent. The water solubility (at room temperature and optimal pH) is at least about 0.05 mg/ml. In some embodiments, the solubility is about 0.1 to about 10 mg/ml or about 1 to about 5 mg/ml.

In some embodiments of formula I, Y is a radical containing at least one acidic group or salt thereof. Such a photoactivatable cross-linking agent can be anionic depending upon the pH of the coating composition. Suitable acidic groups include, for example, sulfonic acids, carboxylic acids, phosphonic acids, and the like. Suitable salts of such groups include, for example, sulfonate, carboxylate, and phosphate salts. In some embodiments, the ionic cross-linking agent includes a sulfonic acid or sulfonate group. Suitable counter ions include alkali, alkaline earths metals, ammonium, protonated amines, and the like.

For example, a compound of formula I can have a radical Y that contains a sulfonic acid or sulfonate group; X₁ and X₂ can contain photoreactive groups such as aryl ketones. Such compounds include 4,5-bis(4-benzoylphenylmethyleneoxy)benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, and the like. See U.S. Pat. No. 6,278,018. The counter ion of the salt can be, for example, ammonium or an alkali metal such as sodium, potassium, or lithium.

In other embodiments of formula I, Y can be a radical that contains a basic group or a salt thereof. Such Y radicals can include, for example, an ammonium, a phosphonium, or a sulfonium group. The group can be neutral or positively charged, depending upon the pH of the coating composition. In some embodiments, the radical Y includes an ammonium group. Suitable counter ions include, for example, carboxylates, halides, sulfate, and phosphate. For example, compounds of formula I can have a Y radical that contains an ammonium group; X₁ and X₂ can contain photoreactive groups that include aryl ketones. Such photoactivatable cross-linking agents include ethylenebis(4-benzoylbenzyldimethylammonium) salt; hexamethylenebis(4-benzoylbenzyldimethylammonium) salt; 1,4-bis(4-benzoylbenzyl)-1,4-dimethylpiperazinediium) salt, bis(4-benzoylbenzyl) hexamethylenetetraminediium salt, bis[2-(4-benzoylbenzyldimethylammonio)ethyl]-4-benzoylbenzylmethylammonium salt; 4,4-bis(4-benzoylbenzyl) morpholinium salt; ethylenebis[(2-(4-benzoylbenzyldimethylammonio)ethyl)-4-benzoylbenzylmethylammonium] salt; and 1,1,4,4-tetrakis(4-benzoylbenzyl)piperzinediium salt. See U.S. Pat. No. 5,714,360. The counter ion is typically a carboxylate ion or a halide. On one embodiment, the halide is bromide.

In other embodiments, the ionic photoactivatable cross-linking agent can be a compound having the formula:

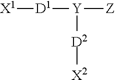

wherein X¹ includes a first photoreactive group; X² includes a second photoreactive group; Y includes a core molecule; Z includes at least one charged group; D¹ includes a first degradable linker; and D² includes a second degradable linker. Additional exemplary degradable ionic photoactivatable cross-linking agents are described in US Patent Application Publication US 2011/0144373 (Swan et al., “Water Soluble Degradable Crosslinker”), the disclosure of which is incorporated herein by reference.

In some aspects a non-ionic photoactivatable cross-linking agent can be used. In one embodiment, the non-ionic photoactivatable cross-linking agent has the formula XR₁R₂R₃R₄, where X is a chemical backbone, and R₁, R₂, R₃, and R₄ are radicals that include a latent photoreactive group. Exemplary non-ionic cross-linking agents are described, for example, in U.S. Pat. Nos. 5,414,075 and 5,637,460 (Swan et al., “Restrained Multifunctional Reagent for Surface Modification”). Chemically, the first and second photoreactive groups, and respective spacers, can be the same or different.

In other embodiments, the non-ionic photoactivatable cross-linking agent can be represented by the formula:

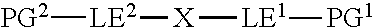

wherein PG¹ and PG² include, independently, one or more photoreactive groups, for example, an aryl ketone photoreactive group, including, but not limited to, aryl ketones such as acetophenone, benzophenone, anthraquinone, anthrone, anthrone-like heterocycles, their substituted derivatives or a combination thereof; LE¹ and LE² are, independently, linking elements, including, for example, segments that include urea, carbamate, or a combination thereof; and X represents a core molecule, which can be either polymeric or non-polymeric, including, but not limited to a hydrocarbon, including a hydrocarbon that is linear, branched, cyclic, or a combination thereof; aromatic, non-aromatic, or a combination thereof; monocyclic, polycyclic, carbocyclic, heterocyclic, or a combination thereof; benzene or a derivative thereof; or a combination thereof. Other non-ionic crosslinking agents are described, for example, in U.S. application Ser. No. 13/316,030 filed Dec. 9, 2011 (Publ. No. US 2012/0149934) (Kurdyumov, “Photocrosslinker”), the disclosure of which is incorporated herein by reference.

Further embodiments of non-ionic photoactivatable cross-linking agents can include, for example, those described in U.S. Provisional Application 61/494,724 filed Jun. 8, 2011 (now U.S. application Ser. No. 13/490,994) (Swan et al., “Photo-Vinyl Primers/Crosslinkers”), the disclosure of which is incorporated herein by reference. Exemplary cross-linking agents can include non-ionic photoactivatable cross-linking agents having the general formula R¹—X—R², wherein R¹ is a radical comprising a vinyl group, X is a radical comprising from about one to about twenty carbon atoms, and R² is a radical comprising a photoreactive group.

Some suitable cross-linking agents are those formed by a mixture of the chemical backbone molecule (such as pentaerythritol) and an excess of a derivative of the photoreactive group (such as 4-bromomethylbenzophenone). An exemplary product is tetrakis(4-benzoylbenzyl ether) of pentaerythritol (tetrakis(4-benzoylphenylmethoxymethyl) methane). See U.S. Pat. Nos. 5,414,075 and 5,637,460.

A single photoactivatable cross-linking agent or any combination of photoactivatable cross-linking agents can be used in forming the coating. In some embodiments, at least one nonionic cross-linking agent such as tetrakis(4-benzoylbenzyl ether) of pentaerythritol can be used with at least one ionic cross-linking agent. For example, at least one non-ionic photoactivatable cross-linking agent can be used with at least one cationic photoactivatable cross-linking agent such as an ethylenebis(4-benzoylbenzyldimethylammonium) salt or at least one anionic photoactivatable cross-linking agent such as 4,5-bis(4-benzoyl-phenylmethyleneoxy)benzene-1,3-disulfonic acid or salt. In another example, at least one nonionic cross-linking agent can be used with at least one cationic cross-linking agent and at least one anionic cross-linking agent. In yet another example, a least one cationic cross-linking agent can be used with at least one anionic cross-linking agent but without a non-ionic cross-linking agent.

An exemplary cross-linking agent is disodium 4,5-bis[(4-benzoylbenzyl)oxy]-1,3-benzenedisulfonate (DBDS). This reagent can be prepared by combining 4,5-Dihydroxylbenzyl-1,3-disulfonate (CHBDS) with 4-bromomethylbenzophenone (BMBP) in THF and sodium hydroxide, then refluxing and cooling the mixture followed by purification and recrystallization (also as described in U.S. Pat. No. 5,714,360, incorporated herein by reference).

A further exemplary cross-linking agent is ethylenebis(4-benzoylbenzyldimethylammonium) dibromide. This agent can be prepared as described in U.S. Pat. No. 5,714,360, the content of which is herein incorporated by reference.

Further cross-linking agents can include the cross-linking agents described in U.S. Publ. Pat. App. No. 2010/0274012 and U.S. Pat. No. 7,772,393 the content of all of which is herein incorporated by reference.

In some embodiments, cross-linking agents can include boron-containing linking agents including, but not limited to, the boron-containing linking agents disclosed in U.S. 61/666,516, entitled “Boron-Containing Linking Agents” by Kurdyumov et al., the content of which is herein incorporated by reference. By way of example, linking agents can include borate, borazine, or boronate groups and coatings and devices that incorporate such linking agents, along with related methods. In an embodiment, the linking agent includes a compound having the structure (I):

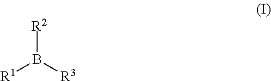

wherein R¹ is a radical comprising a photoreactive group; R² is selected from OH and a radical comprising a photoreactive group, an alkyl group and an aryl group; and R³ is selected from OH and a radical comprising a photoreactive group. In some embodiments the bonds B—R¹, B—R² and B—R³ can be chosen independently to be interrupted by a heteroatom, such as O, N, S, or mixtures thereof.

### Medical Devices

It will be appreciated that embodiments herein include, and can be used in conjunction with, various types of medical devices including, but not limited to, both short-term use (less than 24 hours) and long-term use (greater than or equal to 24 hours) medical devices. Exemplary short-term use devices can include, but are not limited to, cannulae and introducer sheaths, electrophysiology catheters, embolic protection devices, neurological guide wires and catheters, thermodilution catheters, and thromboectomy devices. Exemplary long-term use devices can include, but are not limited to, blood sensors, glucose sensors, central venous catheters, drainage catheters, extracorporeal therapy devices, heart valves, hemodialysis catheters and equipment, implantable pacing and defibrillation devices, oxygenators, peripherally inserted central catheters (PICCs), septal defect repair devices, stimulation leads, temperature management catheters, vascular stents and grafts, vena cava filters, and ventricular assist devices. Devices herein can specifically include neurovascular devices, including but not limited to neurovascular stents, catheters, and the like. Devices herein can also include flow dividers, embolization devices, heart pumps, or other temporary or implanted flow devices.

Embodiments herein can specifically include medical devices with one or more thromboresistant surfaces.

### Methods

Embodiments herein include a variety of methods, including but not limited to, methods of making, methods of using, and the like. It is noted that system or device functionalities detailed elsewhere in this document may be executed as part of one or more methods consistent with the embodiments described herein.

Exemplary embodiments include a method for applying a coating to a medical device. This method involves applying a hydrophilic layer atop a substrate via an in-solution coating process, where the hydrophilic layer contains a photo-PVP compound. Following this, a heparin layer, composed of photo-heparin, is applied over the hydrophilic layer. The combined thickness of these coating layers is generally maintained at or below 100 nanometers.

For certain applications, a tie layer is deposited over the substrate prior to depositing the hydrophilic layer to further enhance the adhesion and integrity of the subsequent layers. As such, in some embodiments, methods herein can incorporate the application of a tie layer onto the substrate before the hydrophilic layer is applied. In certain embodiments, the substrate material is either a metal or a polymer.

The in-solution coating process can include submerging the substrate in a solution containing the photo-PVP compound at a concentration equal to or less than 20 mg/ml and applying UV radiation during the immersion. As demonstrated in the examples, the concentration of the photo-PVP compound in the solution can be significant for achieving the desired layer thickness and properties. In addition, low concentration PVP solutions prevent bulk gelling from occurring with in-solution coating techniques where UV radiation is applied while the substrate is submerged in the coating solution. In various embodiments, the concentration of the photo-PVP compound in the solution ranges from 0.5 to 15 mg/ml.

For the application of the heparin layer over the hydrophilic layer, a dip coating technique can be employed, utilizing a solution of the photo-heparin with a concentration between 50 to 100 mg/ml. The photo-heparin can have a low photo-load which desirably reduces crosslinking to itself.

However, other application techniques for the heparin layer are also contemplated herein. After dip coating, the substrate can be removed from the heparin solution, dried, and then irradiated with UV radiation. In various embodiments, the substrate can also be rinsed and then dried. The heparin layer may be applied in several coats to achieve the desired thickness.

In some embodiments, methods for coating a medical device herein can include steps of applying a first hydrophilic layer over a substrate using an in-solution coating process, where the first hydrophilic layer contains a photo-PVP compound. This is followed by the application of a heparin layer, composed of photo-heparin, over the first hydrophilic layer, and then applying a second hydrophilic layer, also containing a photo-PVP compound, using an in-solution coating process. The total thickness of these coating layers is generally kept at or below 100 nanometers.

The in-solution coating process for both the first and second hydrophilic layers involves immersing the substrate in a solution of the photo-PVP compound at a concentration of 20 mg/ml or less and applying UV radiation during the immersion. In various embodiments, the concentration of the photo-PVP compound in the solution is between 0.5 to 15 mg/ml.

As before, for the application of the heparin layer over the hydrophilic layer, a dip coating technique is utilized, employing a solution of the photo-heparin with a concentration ranging from 50 to 100 mg/ml. However, other application techniques for the heparin layer are also contemplated herein. The heparin layer may also be applied in multiple coats as required.

Aspects may be better understood with reference to the following examples. These examples are intended to be representative of specific embodiments, but are not intended as limiting the overall scope of embodiments herein.

### EXAMPLES

### Example 1: Application of Coating on PEBAX Substate

Compounds used in this example included:
ACRYL-A=PA-BBA-APMA N-Acetylated poly[acrylamide-co-sodium-2-acrylamido-2-methylpropanesulfonate-co-N-(3-(4-benzoylbenzamido)propyl)methacrylamide]
ACRYL-B=PA-BBA-APMA-PEG N-Acetylated poly[acrylamide-co-sodium-2-acrylamido-2-methylpropanesulfonate-co-N-(3-(4-benzoylbenzamido)propyl)methacrylamide]-co-methoxy poly(ethylene glycol) 1000
PVP-A=PVP-APMA-BBA poly[vinyl pyrrolidone^{99%}-co-N-(3-(4-benzoylbenzamideo)propyl)methacrylamide^{1%}]

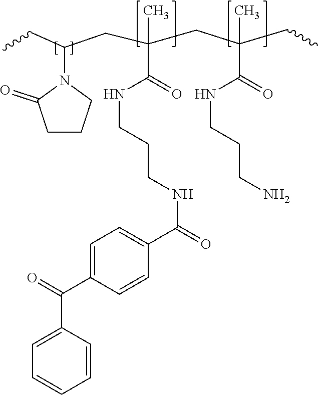

PVP-B=acetylated PVP-APMA-BBA

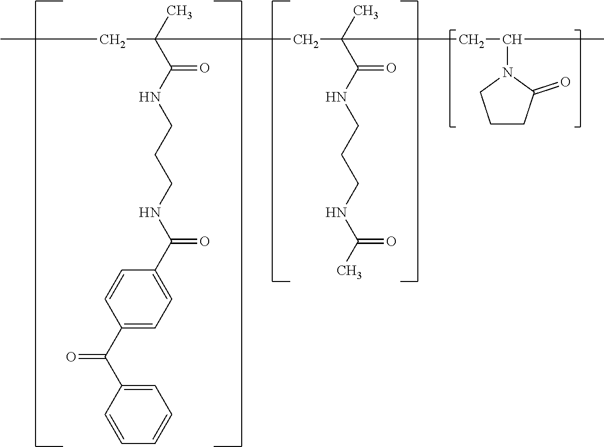

pHEP═photoderivatized heparin
SIL-A=1,4-bis(trimethoxysilylethyl)benzene

A solution of ACRYL-A was prepared in a solvent of 80/20 water/isopropyl alcohol at a concentration of 8 mg/ml.

A solution of ACRYL-B was prepared in a solvent of 80/20 water/isopropyl alcohol at a concentration of 10 mg/ml.

A solution of PVP-A was prepared in a solvent of 80/20 water/isopropyl alcohol at a concentration of 10 mg/ml.

A solution of PVP-B was prepared in a solvent of 80/20 water/isopropyl alcohol at a concentration of 6 mg/ml.

A solution of pHEP in a solvent of 80/20 water/isopropyl alcohol was prepared at a concentration of 80 mg/ml.

PEBAX rods were obtained. The PEBAX rods were first coated with either a solution of ACRYL-A, ACRYL-B, PVP-A, or PVP-B by submerging them in the solution and then applying UV radiation for 30 seconds while they were submerged (an in-solution coating technique).

Then, some of the rods were coated with the pHEP solution by dipping the PEBAX rod into the solution, then removing it and drying, followed by applying UV radiation followed by rinsing. Next, another coat of the pHEP solution was applied in the same way (dip, dry, UV). The resulting PEBAX rods were as shown in Table 1.

TABLE 1

| Sample Code | Coating Configuration |
|---|---|
| B | Uncoated PEBAX Rod |
| 1-1 | ACRYL-A 8 mg/ml |
| 2-1 | ACRYL-B 10 mg/ml |
| 3-1 | PVP-A 10 mg/ml |
| 4-1 | PVP-B 6 mg/ml |
| 1-2 | ACRYL-A 8 mg/ml/pHEP (2 Dip, Dry, UV Coats) |
| 2-2 | ACRYL-B 10 mg/ml/pHEP (2 Dip, Dry, UV Coats) |
| 3-2 | PVP-A 10 mg/ml/pHEP (2 Dip, Dry, UV Coats) |
| 4-2 | PVP-B 6 mg/ml/pHEP (2 Dip, Dry, UV Coats) |

### Example 2: Application of Alternative Coating on PEBAX Substate

PEBAX rods were obtained. In addition to the solutions of the previous example, dilute and very dilute solutions of ACRYL-A, ACRYL-B, PVP-A, and PVP-B were prepared with the same solvents as before, but at concentrations of 5 mg/ml (dilute solutions) and at concentrations of 1 mg/ml (very dilute solutions).

The PEBAX rods were first coated with a normal strength solution of either ACRYL-A, ACRYL-B, PVP-A, or PVP-B using an in-solution coating technique. In specific the PEBAX rods were submerged in the solution and UV radiation was applied for 30 seconds while they were submerged (an in-solution coating technique).

Then, the PEBAX rods were coated with the pHEP solution by dipping them into the pHEP solution, then removing them and allowing them to dry, then exposing them to UV radiation (a dip, dry, UV exposure deposition process). Next, another coat of the pHEP solution was applied in the same way.

Finally, the PEBAX roads were coated with one of ACRYL-A, ACRYL-B, PVP-A, and PVP-B as a dilute solution or a very dilute solution using the in-solution coating technique. The PEBAX rods were then removed from the solution, rinsed, and then allowed to dry. The resulting PEBAX rods were as shown in Table 2.

TABLE 2

| Sample | |
|---|---|
| Code | Coating Configuration |
| 1-3 | In-Solution ACRYL-A/2 coats pHEP/In-Solution ACRYL-A 5 |
| | mg/ml |
| 2-3 | In-Solution ACRYL-B/2 coats pHEP/In-Solution ACRYL-B 5 |
| | mg/ml |
| 3-3 | In-Solution PVP-A/2 coats pHEP/In-Solution PVP-A 5 mg/ml |
| 4-3 | In-Solution PVP-B/2 coats pHEP/In-Solution PVP-B 5 mg/ml |
| 1-4 | In-Solution ACRYL-A/2 coats pHEP/In-Solution ACRYL-A 1 |
| | mg/ml |
| 2-4 | In-Solution ACRYL-B/2 coats pHEP/In-Solution ACRYL-B 1 |
| | mg/ml |
| 3-4 | In-Solution PVP-A/2 coats pHEP/In-Solution PVP-A 1 mg/ml |
| 4-4 | In-Solution PVP-B/2 coats pHEP/In-Solution PVP-B 1 mg/ml |

### Example 3: Thromboresistance Blood Loop Evaluation of Coatings

A 5 vol % solution of SIL-A was prepared in a solvent of acetone.

The SIL-A solution was then applied onto a metal stent forming a tie layer. Next, a PVP-B solution at 6 mg/ml was applied in-solution onto the tie layer. Next, three coats of a pHEP solution at 75 mg/ml 20% IPA/80% water, with rinsing between coats, was applied using a dip, dry, UV process onto the PVP-B layer.

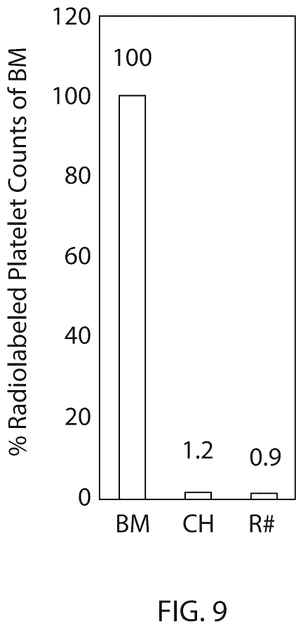

The resulting stents were then evaluated in a blood loop assay for thromboresistance properties and compared with bare metal stents. The results are shown in FIG. 9, which shows percent radiolabeled platelet counts relative to bare metal stents. As can be seen, the tested coatings showed excellent thromboresistance. The tested coatings were also evaluated for heparin activity in the blood loop assay and found to have extremely high levels of heparin activity (greater than 75 mU/cm²).

### Example 4: Fibrinogen Absorbance Evaluation of Coatings

Fibrinogen absorbance properties of the PEBAX rods prepared in Examples 1 and 2 were then evaluated.

In specific, samples were incubated in an aqueous solution of bovine fibrinogen, washed, and fibrinogen adsorption was detected using an anti-fibrinogen HRP-conjugated antibody and a chromogenic substrate. The amount of color change is proportional to the amount of antibody, which is proportional to the amount of adsorbed fibrinogen.

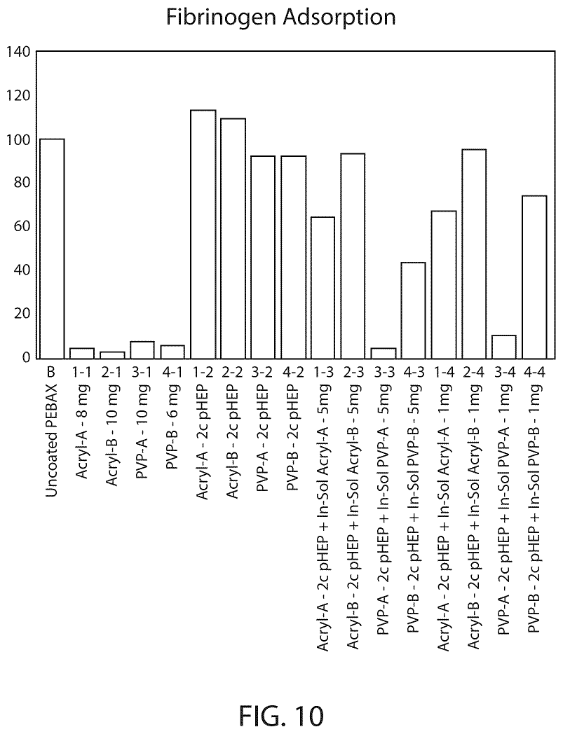

The results are shown in FIG. 10. Lower values reflect less fibrinogen absorption and enhanced thromboresistance. When fibrinogen (FGN) adsorbs to a medical device surface, FGN can denature and cause platelets to attach to the surface and become activated (platelets have a FGN receptor and when bound to denatured FGN the platelets will release contents from their granules which include thrombin, i.e., thrombosis effects).

As can be seen, the presence of in-solution deposited ACRYL-A, ACRYL-B, PVP-A, and PVP-B enhanced thromboresistance properties. In particular, the use of PVP-A as an in-solution deposited topcoat showed remarkable enhancement of thromboresistance properties.

This example shows that the deposition of ACRYL-A, ACRYL-B, PVP-A, and PVP-B in-solution from dilute and very dilute solutions in accordance with embodiments herein can be used to enhance thromboresistance properties of coatings.

Heparin activity was also assessed for the coatings. While deposition of ACRYL-A, ACRYL-B, PVP-A, and PVP-B in-solution over pHEP decreased heparin activity slightly versus controls, heparin activity remained high (greater than or equal to 24 mU/cm²).

These examples are intended to be representative of specific embodiments but are not intended as limiting the overall scope of embodiments herein.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration.

The phrase “configured” can be used interchangeably with other similar phrases such as arranged and configured, constructed, and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. The medical device of claim 10, further comprising:
a tie layer, wherein the tie layer is disposed over the substrate; and
wherein the first hydrophilic layer is disposed over the tie layer.

2. (canceled)

3. The medical device of claim 1, the tie layer comprising at least one selected from the group consisting of a silane compound and a phosphonate compound.

4-7. (canceled)

8. The medical device of claim 1, the tie layer comprising 1,4-bis(trimethoxysilylethyl)benzene.

9. (canceled)

10. A medical device with an antithrombogenic coating comprising:
a substrate; and
a first hydrophilic layer, the first hydrophilic layer comprising a first photo-PVP;
a first heparin layer, wherein the first heparin layer is disposed over the first hydrophilic layer, the first heparin layer comprising
a first photo-heparin; and
wherein the first hydrophilic layer and the first heparin layer are less than or equal to 100 nm thick in total.

11. The medical device of claim 10, wherein the substrate is a metal or a polymer.

12. The medical device of claim 10, the first photo-PVP comprising acetylated PVP-APMA-BBA.

13. The medical device of claim 10, the first hydrophilic layer further comprising sodium bis(4-benzoylphenyl phosphate) and acetylated PVP-APMA-BBA.

14. The medical device of claim 10, wherein the first heparin layer includes at least two coats of the photo-heparin.

15. The medical device of claim 10, wherein the first heparin layer includes at least three coats of the photo-heparin.

16. The medical device of claim 10, wherein the medical device is a neurovascular stent.

17. The medical device of claim 10, further comprising:
a second hydrophilic layer, the second hydrophilic layer comprising a second photo-PVP;
wherein the second hydrophilic layer is disposed over the first heparin layer;
a second heparin layer, the second heparin layer comprising a second photo-heparin;
wherein the second heparin layer is disposed over the second hydrophilic layer; and
wherein the tie layer, the first hydrophilic layer, the first heparin layer, the second hydrophilic layer, and the second heparin layer are less than or equal to 100 nm thick in total.

18-22. (canceled)

23. The medical device of claim 10, wherein:
wherein the first heparin layer is disposed over the first hydrophilic layer and further comprises
a photo-PVP; and
wherein the first hydrophilic layer, and the first heparin layer containing photo-PVP are less than or equal to 100 nm thick in total.

24-28. (canceled)

29. The medical device of claim 1 further comprising:
wherein the hydrophilic layer further comprises:
iodine.

30-47. (canceled)

48. The medical device of claim 10, wherein the coating exhibits enhanced heparin activity and low fibrinogen binding.

49. The medical device of claim 10 further comprising:
a second hydrophilic layer, wherein a second hydrophilic layer is disposed over the heparin layer, the second hydrophilic layer comprising
a second photo-PVP.

50. The medical device of claim 49, wherein the second hydrophilic layer is deposited by applying UV radiation while the medical device is immersed in a solution of the second photo-PVP and a solvent.

51. The medical device of claim 50, wherein a solution of the second photo-PVP and a solvent has a concentration of the second photo-PVP of between 0 mg/ml and 10 mg/ml.

52-53. (canceled)

54. The medical device of claim 49, the second photo-PVP comprising PVP-APMA-BBA.

55. (canceled)

56. The medical device of claim 49, wherein the hydrophilic layer, the heparin layer, and the second hydrophilic layer are less than or equal to 100 nm thick in total.

57-88. (canceled)
